(19) United States
(12) Patent Application Publication
(10) Pub. No.: US 2006/0210798 A1
(43) Pub. Date: Sep. 21, 2006

(54) Doped metal oxide nanoparticles and methods for making and using same

(76) Inventor: Clemens Burda, Cleveland Heights, OH (US)

Correspondence Address: BRINKS HOFER GILSON & LIONE, P.O. BOX 10395, CHICAGO, IL, 60610, US

(21) Appl. No.: 11/081,862

(22) Filed: Mar. 16, 2005

(51) Int. Cl. B32B 5/16; B01J 21/18; B01J 21/02; B01J 27/00; B01J 27/057; B01J 27/02; B01J 27/06; B01J 27/135; C01G 23/047; A61K 33/24

(52) U.S. Cl. 428/402; 428/403; 502/182; 502/202; 502/208; 502/215; 502/216; 502/224; 502/227; 423/610; 588/300; 424/617; 588/306; 588/408; 977/906

(57) ABSTRACT

Metal oxide nanoparticles are described that contain a non-metallic dopant selected from the group consisting of boron, carbon, silicon, germanium, nitrogen, phosphorous, arsenic, sulfur, selenium, tellurium, fluorine, chlorine, bromine, iodine, and combinations thereof. Methods of making and using these doped metal oxide nanoparticles are also described.

### FEDERALLY SPONSORED RESEARCH OR DEVELOPMENT

This invention was made in part with Government support (Grant No. CHE-0239688 awarded by the National Science Foundation). The Government may have certain rights in this invention.

### FIELD OF THE INVENTION

The present invention relates to doped metal oxide nanoparticles and, more particularly, to metal oxide nanoparticles doped with one or more non-metal elements.

### BACKGROUND

The efficient utilization of solar energy represents a long-standing goal of modern science and engineering with potential applications existing across a broad spectrum of technologies. Titanium dioxide (TiO₂), also known as titania, is one of the most promising materials being developed for photocatalytic applications due to its low cost, photostability, chemical inertness, and high efficiency.

Unfortunately, the wide band gap of titanium dioxide (3.2 eV) greatly limits its use as a photocatalyst because light from the ultraviolet (UV) region of the spectrum is required for its activation. Since UV light represents only a small fraction of solar light (about 8%), it would be highly advantageous to shift the optical response of titanium dioxide towards the visible region of the spectrum, which accounts for a much higher fraction of solar light (about 45%). Moreover, shifting the optical response of titanium dioxide from UV light towards visible light would greatly increase the photocatalytic efficiency of the material.

One approach that has been tried for shifting the optical response of titanium dioxide to the visible range has been doping the material with transition metal elements. However, metal doping has several drawbacks, including the thermal instability of the metal doped material, and the fact that the metal centers acts as electron traps and may reduce the photocatalytic efficiency of the doped material. In addition, the preparation of transition metal doped titanium dioxide typically requires the use of ion-implantation facilities, which significantly increases the expense of the process.

### SUMMARY

The scope of the present invention is defined solely by the appended claims, and is not affected to any degree by the statements within this summary.

A first material embodying features of the present invention includes one or a plurality of titanium dioxide nanoparticles containing a non-metallic dopant selected from the group consisting of boron, carbon, silicon, germanium, phosphorous, arsenic, sulfur, selenium, tellurium, fluorine, chlorine, bromine, iodine, and combinations thereof.

A second material embodying features of the present invention includes one or a plurality of titanium dioxide nanoparticles containing a non-metallic dopant selected from the group consisting of boron, carbon, silicon, germanium, phosphorous, arsenic, sulfur, selenium, tellurium, fluorine, chlorine, bromine, iodine, and combinations thereof. In this material, the nanoparticles include an average diameter ranging from about 0.5 nm to about 350 nm, and the nanoparticles include from about 0.1 percent to about 15 percent of the non-metallic dopant. At least a portion of the nanoparticles absorb visible light.

A third material embodying features of the present invention includes one or a plurality of transition metal oxide nanoparticles, wherein the transition metal is selected from the group consisting of zirconium, hafnium, group VA metals, group VIA metals, group VIIA metals, group VIIIA metals, group IB metals, group IIB metals, and combinations thereof. The transition metal oxide nanoparticles comprise a non-metallic dopant selected from the group consisting of boron, carbon, silicon, germanium, nitrogen, phosphorous, arsenic, sulfur, selenium, tellurium, fluorine, chlorine, bromine, iodine, and combinations thereof.

A first method of making doped titanium dioxide nanoparticles embodying features of the present invention includes hydrolyzing Ti(OR)₄ in the presence of a dopant selected from the group consisting of boron, carbon, silicon, germanium, phosphorous, arsenic, sulfur, selenium, tellurium, fluorine, chlorine, bromine, iodine, and combinations thereof to form doped titanium dioxide nanoparticles. R is an alkyl group.

A second method of making doped titanium dioxide nanoparticles embodying features of the present invention includes oxidizing a powder having a formula TiX, wherein X is selected from the group consisting of boron, carbon, silicon, germanium, nitrogen, phosphorous, arsenic, sulfur, selenium, tellurium, fluorine, chlorine, bromine, iodine, and combinations thereof to form titanium dioxide nanoparticles doped with X.

A first doped titanium dioxide nanoparticle embodying features of the present invention includes a core portion, a shell portion, and a non-metallic dopant, wherein the core portion is adjacent to a center of the nanoparticle and the shell portion is adjacent to an exterior surface of the nanoparticle. The non-metallic dopant is selected from the group consisting of boron, carbon, silicon, germanium, phosphorous, arsenic, sulfur, selenium, tellurium, fluorine, chlorine, bromine, iodine, and combinations thereof. A concentration of the non-metallic dopant is higher in the shell portion than in the core portion.

A second doped titanium dioxide nanoparticle embodying features of the present invention includes a core portion, a shell portion, and a non-metallic dopant, wherein the core portion is adjacent to a center of the nanoparticle and the shell portion is adjacent to an exterior surface of the nanoparticle. The non-metallic dopant is selected from the group consisting of boron, carbon, silicon, germanium, nitrogen, phosphorous, arsenic, sulfur, selenium, tellurium, fluorine, chlorine, bromine, iodine, and combinations thereof. A concentration of the non-metallic dopant is higher in the core portion than in the shell portion.

A composition for covering a surface embodying features of the present invention includes a solvent, one or a plurality of pigments, and one or a plurality of titanium dioxide nanoparticles containing a non-metallic dopant selected from the group consisting of boron, carbon, silicon, germanium, nitrogen, phosphorous, arsenic, sulfur, selenium, tellurium, fluorine, chlorine, bromine, iodine, and combinations thereof.

A method of treating an environmental contaminant embodying features of the present invention includes (a) providing one or a plurality of titanium dioxide nanoparticles containing a non-metallic dopant selected from the group consisting of boron, carbon, silicon, germanium, nitrogen, phosphorous, arsenic, sulfur, selenium, tellurium, fluorine, chlorine, bromine, iodine, and combinations thereof; (b) activating at least a portion of the nanoparticles with visible light to form activated nanoparticles; and (c) oxidizing at least a portion of the environmental contaminant by reaction with the activated nanoparticles.

A method of catalyzing a chemical reaction with sunlight embodying features of the present invention includes (a) providing one or a plurality of titanium dioxide nanoparticles containing a non-metallic dopant selected from the group consisting of boron, carbon, silicon, germanium, nitrogen, phosphorous, arsenic, sulfur, selenium, tellurium, fluorine, chlorine, bromine, iodine, and combinations thereof; (b) activating at least a portion of the nanoparticles with sunlight to form activated nanoparticles; and (c) catalyzing a chemical reaction with the activated nanoparticles.

A method of treating a patient having cancer embodying features of the present invention includes (a) providing one or a plurality of titanium dioxide nanoparticles containing a non-metallic dopant selected from the group consisting of boron, carbon, silicon, germanium, nitrogen, phosphorous, arsenic, sulfur, selenium, tellurium, fluorine, chlorine, bromine, iodine, and combinations thereof; (b) activating at least a portion of the nanoparticles with light to form activated nanoparticles; (c) transferring energy from at least a portion of the activated nanoparticles to an oxygen molecule to form a reactive singlet oxygen; and (d) reacting the reactive singlet oxygen with a cancer cell thereby destroying the cancer cell.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows an XPS spectrum of a TiO₂₋ₓNₓ nanoparticle sample with an average diameter of 10 nm measured on a carbon support.

FIG. 2 shows plots of reflectance measurements demonstrating the red shift in optical response caused by the nitrogen doping of TiO₂ nanoparticles.

FIG. 3A shows a comparison of the photocatalytic decomposition of methylene blue in the presence of doped and undoped titanium dioxide nanoparticles, as monitored by the changes in absorbance at 650 nm after 390-nm laser excitation. The inset shows the photodegradation of methylene blue in water at neutral pH.

FIG. 3B shows a comparison of the photocatalytic decomposition of methylene blue in the presence of doped and undoped titanium dioxide nanoparticles, as monitored by the changes in absorbance at 650 nm after 540-nm excitation.

FIG. 4A shows XRD patterns of TiC powder (black line) and the resultant TiO₂ powders (red line) after heating at 350° C. for 96 hours, demonstrating that micrometer sized TiC powder was completely converted into TiO₂ nanocrystals.

FIG. 4B shows reflectance spectra of TiC powder (black line) and the resultant TiO₂ nanocrystals after heating (red line), with the blue dashed lines indicating the band gap energy for the resultant TiO₂ nanocrystals.

FIG. 5 shows images of micrometer-sized TiC powders and the TiO₂ nanocrystals that are produced by controlled oxidation of the starting material.

FIG. 6 shows the C 1s core-level XPS pattern of commercial TiC powder.

FIG. 7 shows the C 1s core-level XPS pattern of TiO₂ nanocrystals obtained by heating commercial TiC powder.

FIG. 8 shows the Ti 2p core-level XPS pattern of TiO₂ nanocrystals obtained by heating commercial TiC powder.

FIG. 9 shows the Raman spectrum of TiO₂ nanocrystals obtained by heating commercial TiC powder.

FIG. 10 shows TEM images of TiC powder and the resultant TiO₂ nanoparticles obtained after heating.

FIG. 11 shows HRTEM images of the TiO₂ nanocrystals obtained from TiC after heating.

FIG. 12 shows XRD patterns of TiN powder and the TiO₂ nanocrystals obtained by heating TiN powder at 650° C. for 96 hours.

FIG. 13 shows reflectance spectra of TiN powder and the TiO₂ nanocrystals obtained by heating TiN powder at 650° C. for 96 hours.

FIG. 14 shows XRD patterns of TiS₂ powder and the TiO₂ nanocrystals obtained by heating TiS₂ powder at 450° C. for 96 hours.

FIG. 15 shows reflectance spectra of TiS₂ powder and the TiO₂ nanocrystals obtained by heating TiS₂ powder at 450° C. for 96 hours.

FIG. 16 shows XPS patterns of the TiO₂ nanocrystals obtained by heating TiC, TiN, and TiS₂.

FIG. 17 shows XRD patterns for (a) commercial TiN, (b) TiN after heating, (c) commercial TiC, (d) TiC after heating, (e) commercial TiS₂, and (f) TiS₂ after heating.

FIG. 18 shows wide energy range XPS spectra of TiN (black line) and the resultant TiO₂ (red line).

FIG. 19 shows reflectance spectra for (a) commercial TiN, (b) TiN after heating, (c) commercial TiC, (d) TiC after heating, (e) commercial TiS₂, and (f) TiS₂ after heating.

FIG. 20 shows XAS spectra of Ti 2p in TiN and the resultant TiO₂ samples.

FIG. 21 shows XAS spectra of O 1s in TiN and the resultant TiO₂ samples.

FIG. 22 shows XES spectra of (a) Ti L and (b) O K_{α} in TiN and the resultant TiO₂ samples.

FIG. 23 shows XPS spectra of (a) Ti 2p and (b) O 1s in TiN and the resultant TiO₂ samples.

FIG. 24 shows XPS spectra and their fittings of the valence band structures for (a) TiN and (b) the resultant TiO₂.

FIG. 25 shows (a) assignment of the O K_{α} x-ray emission spectrum from (b) the transitions between the O 1s core-level and the valence band structure (fitting curve) from XPS measurement, and (c) assignment of the Ti L x-ray emission spectrum from (d) the transitions between the Ti 2p core-level and the valence band structure (fitting curve) from XPS measurement.

FIG. 26 shows a comparison of the sum of the PVBs of O and Ti to the total valence band (VB) from XPS measurement with its fitting.

FIG. 27 shows (a) the correlation of the O 1s core-level XPS and O 1s x-ray absorption spectra used to construct the partial conduction band (PCB) structure having O 2p characters, and (b) the correlation of Ti 2p core-level XPS and Ti 2p x-ray absorption spectra used to construct the partial conduction band (PCB) structure having Ti 3d characters.

FIG. 28 shows the constructed conduction band (CB) and the partial conduction bands (PCBs) from O and Ti in the TiO₂ obtained from TiN.

FIG. 29 shows (A) comparison of the inverse-photoemission spectrum (a) and theoretical calculation of the band structure (b) of TiO₂ to the constructed conduction band structure (c), and (B) comparison of the constructed band structures of the resultant TiO₂ from TiN in this contribution to the theoretical single particle calculations of the band structures of TiO₂

FIG. 30 shows XRD patterns of Degussa P25 TiO₂ powder (black line) and nitrogen-doped TiO₂ nanocatalyst before calcination (red) and after calcination at 400° C. (green).

FIG. 31 shows TEM images of nitrogen-doped TiO₂ nanocatalyst before (left) and after (right) calcinations with the bar scale in the TEM set at 50 nm.

FIG. 32 shows plots of the decolorization of AO7 with three different nanoparticles.

FIG. 33 shows plots of the decolorization of azo dyes by TiO₂ nanoparticles.

FIG. 34 shows plots of the decolorization of azo dyes by N-doped TiO₂ nanoparticles.

FIG. 35 shows plots of the TOC disappearance of azo dyes by N-doped TiO₂ nanoparticles.

FIG. 36 shows plots of SO₄²⁻ generation of azo dyes by N-doped TiO₂ nanoparticles.

### DETAILED DESCRIPTION

Metal oxide nanoparticles doped with one or a plurality of non-metallic main group elements have been discovered and are described hereinbelow. In some embodiments, the metal oxide is titanium dioxide. The doped nanoparticles that have been discovered and are described below are photocatalytically active and have absorptions that extend into the visible region of the spectrum. Methods of manufacturing these metal oxide nanoparticles have also been discovered and are described below. Depending on the particular method used, the concentration gradient of dopant in the nanoparticle may be controlled and varied, such that nanoparticles having characteristics tailor-made for specific applications may be prepared. Methods of using the metal oxide nanoparticles have also been discovered and are described below.

Throughout this description and in the appended claims, the following definitions are to be understood:

The term “nanoparticle” refers to a particle that exhibits one or more properties not normally associated with a corresponding bulk material (e.g., quantum optical effects, etc.). As used herein, the term usually refers to materials having nanometer-sized dimensions that do not exceed about 1000 nm (although in many embodiments these dimensions are even smaller). In some embodiments, the nanoparticles are in a crystalline state and the term “nanocrystal” may be used interchangeably with “nanoparticle.” In some embodiments, the nanoparticles may be used in photocatalytic applications, and the term “nanoparticle” may be used interchangeably with “nanocatalyst.”

The terms “main group,” “transition metal,” “group IVA metals,” “group VA metals,” “group VIA metals,” “group VIIA metals,” “group VIIIA metals,” “group IB metals,” and “group IIB metals” refer to elements as they are grouped in the Periodic Table of the Elements. The subgroup designations A and B refer to the designations recommended by the International Union of Pure and Applied Chemistry (IUPAC).

The phrase “alkyl group” refers to any straight, branched, cyclic, acyclic, saturated or unsaturated carbon chain. Representative alkyl groups include but are not limited to alkanes, alkenes, alkynes, cycloalkanes, cycloalkenes, cycloalkynes, aryls, and the like, and combinations thereof.

The term “core” or “core portion” refers to an interior or central region of a nanoparticle adjacent to its center. By analogy, the term “shell” or “shell portion” refers to a region of the nanoparticle that substantially surrounds the core and which is adjacent to and may include the exterior surface of the nanoparticle.

By way of introduction, a material embodying features of the present invention includes one or a plurality of transition metal oxide nanoparticles, wherein the transition metal is selected from the group consisting of group IVA metals, group VA metals, group VIA metals, group VIIA metals, group VIIIA metals, group IB metals, group IIB metals, and combinations thereof. The transition metal oxide nanoparticles are doped with a non-metallic dopant selected from the group consisting of boron, carbon, silicon, germanium, nitrogen, phosphorous, arsenic, sulfur, selenium, tellurium, fluorine, chlorine, bromine, iodine, and combinations thereof.

In some embodiments, the transition metal is a group IVA metal, which in some embodiments is titanium. In these embodiments, the transition metal oxide corresponds to titanium dioxide.

In some embodiments, the transition metal is chromium, molybdenum, tungsten, manganese, iron, cobalt, nickel, palladium, platinum, zinc or cadmium. In some embodiments, the transition metal oxide is selected from the group consisting of ZnO, Fe₂O₃, and WO₃.

When the transition metal is titanium, a material embodying features of the present invention includes one or a plurality of titanium dioxide nanoparticles comprising a non-metallic dopant selected from the group consisting of boron, carbon, silicon, germanium, nitrogen, phosphorous, arsenic, sulfur, selenium, tellurium, fluorine, chlorine, bromine, iodine, and combinations thereof.

In some embodiments, the non-metallic dopant is selected from the group consisting of boron, carbon, silicon, nitrogen, phosphorous, sulfur, selenium, fluorine, chlorine, bromine, and combinations thereof. In other embodiments, the non-metallic dopant is selected from the group consisting of carbon, silicon, nitrogen, phosphorous, sulfur, fluorine, chlorine, and combinations thereof.

In some embodiments, the average diameter of doped metal oxide nanoparticles embodying features of the present invention ranges from about 0.1 nm to about 1000 nm. In some embodiments, the average diameter ranges from about 0.3 nm to about 500 nm. In some embodiments, the average diameter ranges from about 0.5 nm to about 350 nm. In some embodiments, the average diameter ranges from about 1 nm to about 200 nm.

In some embodiments, doped metal oxide nanoparticles embodying features of the present invention contain from about 0.05 to about 20 percent of the non-metallic dopant. In some embodiments, the nanoparticles contain from about 0.1 percent to about 15 percent of the non-metallic dopant. In some embodiments, the nanoparticles contain from about 0.5 percent to about 12 percent of the non-metallic dopant. In some embodiments, the nanoparticles contain from about 1 percent to about 10 percent of the non-metallic dopant. In some embodiments, the nanoparticles contain from about 4 percent to about 8 percent of the non-metallic dopant.

In some embodiments, doped metal oxide nanoparticles embodying features of the present invention absorb light in the visible region of the electromagnetic spectrum (i.e., light having a wavelength ranging from about 380 nm to about 780 nm). In some embodiments, the nanoparticles absorb light having a wavelength of at least about 390 nm. In some embodiments, the nanoparticles absorb light having a wavelength of at least about 450 nm. In some embodiments, the nanoparticles absorb light having a wavelength of at least about 500 nm. In some embodiments, the nanoparticles absorb light having a wavelength of at least about 550 nm.

It has been discovered that the wavelength of light absorbed by a metal oxide nanoparticle embodying features of the present invention varies with the size of the nanoparticle and the concentration of dopant. In general, larger particles absorb at longer wavelengths than smaller particles. In addition, lower concentrations of dopant correlate with absorption at shorter wavelengths while higher concentrations of dopant correlate with absorption at longer wavelengths.

In some embodiments, metal oxide nanoparticles embodying features of the present invention further include a metallic dopant in addition to the non-metallic dopant. In some embodiments, the metallic dopant is a transition metal.

Metal capping is a technique that has been explored for shifting the absorption of metal oxides (e.g., titanium dioxide) to the visible region of the spectrum. By applying a metal on the surface of a typically porous surface (e.g., by using a technique such as sputtering), a substantially unbroken outer metal shell or, alternatively, one or more metallic “nano-islands” may be formed on the surface of the nanoparticle, which may be advantageous for use in catalytic applications.

Thus, in some embodiments, metal oxide nanoparticles embodying features of the present invention further include a metal cap on at least a portion of the outer surface of the nanoparticle. In some embodiments, the metal cap is a transition group metal. In some embodiments, one or more different transition group metals are applied as metal caps on the same nanoparticle. In some embodiments, the transition metals used as metal caps are selected from the group consisting of ruthenium, rhodium, nickel, palladium, platinum, copper, gold, silver, and combinations thereof.

As further described below, the concentration profile of dopant in a metal oxide nanoparticle embodying features of the present invention may be controlled and varied according to the specific method by which the material is prepared. In such a way, nanoparticles embodying features of the present invention may be provided as core-shell structures. For purposes of illustration, a core-shell nanoparticle may be thought of as a sphere within a sphere, wherein the innermost sphere corresponds to the core of the nanoparticle and the outermost sphere, which substantially surrounds the core, corresponds to the shell of the nanoparticle. It is to be understood, of course, that the above-described simplified representation of a core-shell structure is intended only as a mnemonic and that the actual structures of the nanoparticles may be more complex (e.g., neither the core nor the shell may be perfectly spherical and one or both may assume all manner of regular or irregular geometric shapes). In some embodiments, the concentration of dopant is higher in the shell than in the core (e.g., nitrogen-doped titanium dioxide nanoparticles prepared by the hydrolysis of titanium tetraisopropoxide in the presence of an amine, further described below). In other embodiments, the concentration of dopant is higher in the core than in the shell (e.g., nitrogen-doped titanium dioxide nanoparticles prepared by the controlled oxidation of TiN, further described below).

Core-shell doped titanium dioxide nanoparticles embodying features of the present invention include a core portion, a shell portion, and a non-metallic dopant, wherein the core portion is adjacent to a center of the nanoparticle and the shell portion is adjacent to an exterior surface of the nanoparticle. The non-metallic dopant is selected from the group consisting of boron, carbon, silicon, germanium, nitrogen, phosphorous, arsenic, sulfur, selenium, tellurium, fluorine, chlorine, bromine, iodine, and combinations thereof. In a first type of core-shell doped titanium dioxide nanoparticles embodying features of the present invention, the concentration of non-metallic dopant is higher in the shell portion than in the core portion. In a second type of core-shell doped titanium dioxide nanoparticles embodying features of the present invention, the concentration of non-metallic dopant is higher in the core portion than in the shell portion.

In some embodiments, the non-metallic dopant of the core-shell nanoparticles is selected from the group consisting of carbon, silicon, nitrogen, phosphorous, sulfur, fluorine, and chlorine. In some embodiments, the non-metallic dopant is nitrogen.

Methods of manufacturing doped metal oxide nanoparticles in accordance with the present invention will now be described.

A first method of making doped titanium dioxide nanoparticles embodying features of the present invention includes hydrolyzing Ti(OR)₄ in the presence of a dopant selected from the group consisting of boron, carbon, silicon, germanium, nitrogen, phosphorous, arsenic, sulfur, selenium, tellurium, fluorine, chlorine, bromine, iodine, and combinations thereof to form doped titanium dioxide nanoparticles. The variable R in Ti(OR)₄ is an alkyl group. In some embodiments, R is selected from the group consisting of —CH₃, —CH₂CH₃, —CH₂CH₂CH₃, —CH(CH₃)₂, and —CH₂CH₂CH₂CH₃. In some embodiments, R is —CH(CH₃)₂.

The hydrolysis-based approach described above for making doped titanium dioxide nanoparticles produces a titanium dioxide network in which dopant atoms are integrated. Nanometer-sized particles correspond to kinetically-controlled products. Thus, the growth of crystals formed by this method is stopped before bulk material is allowed to form. The stoppage may be achieved by quenching the hydrolysis reaction (e.g., diluting the reaction suddenly and/or centrifuging the reaction mixture and removing the particles) before particles larger than about 1 μm begin to form.

Typically, the concentration of dopant in nanocrystals formed with this method is higher in the shell of the nanoparticle than in the core, which renders these materials particularly suitable for use as catalysts. While neither desiring to be bound by any particular theory, nor intending to limit in any measure the scope of the appended claims or their equivalents, it is presently believed that there may be a tendency for dopant to move towards the outside of the nanoparticle over time (i.e., the lattice may not be static), a type of “self-cleaning” process that has previously been observed in connection with manganese dopants.

Further details of the above-described hydrolysis-based approach for making doped titanium dioxide nanoparticles embodying features of the present invention, as well as additional details regarding the properties and spectroscopic investigation of these materials, are provided in the present inventor's journal articles entitled “Enhanced Nitrogen Doping in TiO₂ Nanoparticles” (Nano Letters, 2003, 3, No. 8, 1049-1051), “Highly Efficient Formation of Visible Light Tunable TiO₂₋ₓNₓ Photocatalysts and Their Transformation at the Nanoscale” (J. Phys. Chem. B, 2004, 108, 1230-1240), and “Photoelectron Spectroscopic Investigation of Nitrogen-Doped Titania Nanoparticles” (J. Phys. Chem. B, 2004, 108, 15446-15449). The entire contents of all three of these articles are incorporated herein by reference, except that in the event of any inconsistent disclosure or definition from the present application, the disclosure or definition herein shall be deemed to prevail.

TiO₂₋ₓNₓ nanoparticles may be prepared, for example, by employing the direct amination of 6-10-nm-sized titania particles at room temperature. Doping on the nanometer scale leads to an enhanced nitrogen concentration of up to about 8%, compared to ≦about 2% in thin films and micrometer-scale TiO₂ powders, with higher concentrations also being possible. The TiO₂₋ₓNₓ nanoparticles thus synthesized are photocatalytically active and absorb well into the visible region up to about 600 nm. In addition, the nanocrystals show enhanced efficiency in the photodegradation of methylene blue under visible light irradiation (wavelength≧390 nm) as compared to commercially available TiO₂.

In the past, considerable effort was expended to dope TiO₂ thin films and powders with nitrogen by annealing TiO₂ at elevated temperature under an ammonia flow for several hours. Nevertheless, the doping process on these micrometer-sized TiO₂ systems resulted in only small amounts (≦about 2%) of nitrogen incorporation. By contrast, the hydrolysis-based approach in accordance with the present invention leads to increased nitrogen dopant concentration in titanium dioxide.

In some embodiments, the hydrolysis procedure entails the dropwise addition of titanium isopropoxide solution to an ethanol solution under vigorous stirring. For example, the preparation of N-doped TiO₂ may be accomplished by the slow addition of the Ti precursor into a mixture of water, alcohol, and amines under strong stirring. To facilitate the growth of the nanoparticles, the reaction mixture may be stirred over night at a temperature of about 90° C. Changing the ratio of water/alcohol/amine and/or the Ti precursor concentrations enables the preparation of different sizes of TiO₂ nanoparticles doped with different amounts of nitrogen. Substitution of the amines with other reagents (e.g., NH₄F, H₂S, HCl, etc.) allows incorporation of other main group elements into the TiO₂ lattice.

The doped nanoparticles thus synthesized may then be isolated from solution by vacuum evaporation at room temperature. This step may be followed by heat treatment at, for example, about 300° C. to about 500° C. for about 3 to about 4 hours to convert amorphous nanoparticles to the desired anatase crystallographic phase.

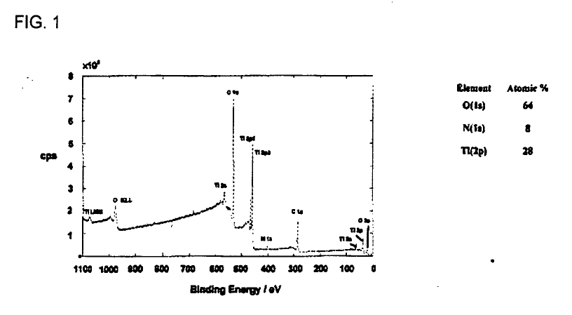

As further described below, small TiO₂ nanocrystals were prepared by the controlled hydrolysis of titanium (IV) isopropoxide in water under controlled pH. By adjusting the pH of the solution, TiO₂ nanocrystals in sizes ranging from about 3 to about 10 nm may be synthesized as transparent colloidal solutions, which are stable for extended periods. To introduce the nitrogen dopant, an amine (e.g., triethylamine) is added to the colloidal nanoparticle solution. The addition of amine to the nanoparticle solution results in the formation of yellow nanocrystals (mean diameter of about 10 nm). X-ray powder diffractometry (XRD) and high-resolution transmission electron microscopy (HRTEM) demonstrate that the treated nanostructures are of the anatase crystalline phase. As shown in FIG. 1, x-ray photoelectron spectroscopy (XPS) confirms enhanced nitrogen incorporation.

The change in color of the nanocrystals upon nitrogen incorporation demonstrates a profound effect on their optical response in the visible wavelength range. With varying degree of nitrogen doping, the color of the nanocrystals changes. In contrast to the nanoparticle reactivity described above, no significant reaction was observed when TiO₂ micropowders were treated with triethylamine. Furthermore, the treatment of Degussa P25 “nanopowder” (particle size≧30 nm) resulted in a much slower doping reaction.

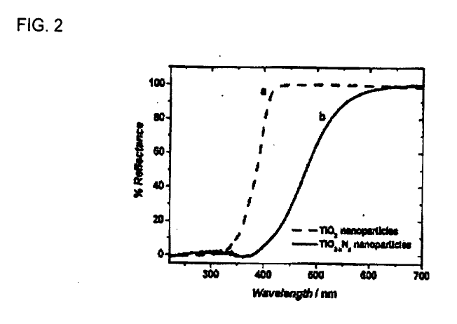

FIG. 2 shows the UV-visible reflectance spectra of pure TiO₂ and nitrogen-doped TiO₂ nanoparticles. The optical reflectance spectrum of commercial Degussa P25 TiO₂ (spectrum a) has an onset at about 380 nm, as compared to the reflectance spectrum for approximately 10 nm-sized TiO₂₋ₓNₓ nanocrystals (spectrum b), which rises at about 600 nm. Thus, the reflectance measurements on the doped TiO₂ nanoparticles show that the band gap absorption onset of the nanocrystals shifted from 380 to 600 nm.

The photocatalytic activity of the nitrogen-doped titanium dioxide nanoparticles prepared as described above was evaluated by measuring the decomposition of methylene blue at 650 nm upon photoexcitation with 390- and 540-nm visible light using a Clark MXR 2001 femtosecond laser system. The laser beam (800 fs, 1 kHz, 120-fs laser pulse train) was sent either through a BBO crystal to generate second harmonic 390-nm (10 mW) light or to an optical parametric amplifier to generate stable 540-nm (4 mW) light. The pulse train was guided into a quartz cuvette filled with 2 mL of an aqueous solution of methylene blue (optical density of 0.8) and 10 mg of the catalyst to excite a pump volume of about 5 nL (0.5 mm is the diameter of the excitation beam at the reaction cell). The decomposition of the solute was followed by measuring the decolorization of the methylene blue in solution with a Varian Cary Bio50 UV-vis spectrometer. Analysis of the monochromatic photon flux taking into account the excitation volume shows that about 1 photon/particle was used for excitation, which is in the low-intensity regime and compares with bright sunlight excitation.

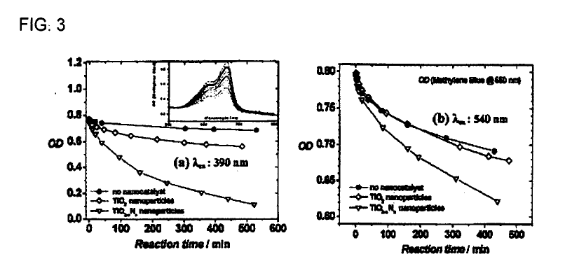

FIG. 3 shows the observed photodegradation of methylene blue in water at neutral pH. The nitrogen-doped titanium dioxide nanoparticles prepared as described above show enhanced photocatalytic activity. However, undoped TiO₂ nanoparticles (Degussa P25) did not show much activity under visible-light radiation as compared to the reference experiment without nanoparticles. The observed gradual decrease in the absorption of methylene blue over time is attributed to the direct decomposition of the dye upon laser irradiation and is not due to the excitation of the Degussa P25 TiO₂ nanocrystals. The photodegradation of methylene blue through the so-called sensitization process in the visible range at wavelengths>500 nm has been observed. Note that the data obtained for TiO₂ as shown in FIG. 3 have a significant component due to the direct photodegradation of methylene blue.

The difference in the photocatalytic activities of the nanoparticles observed after 390- and 540-nm excitation may be correlated with their reflectance spectra shown in FIG. 2. At 390 nm, a larger difference in optical response is observed, which explains the significant difference in the photocatalytic activity at 390-nm irradiation (FIG. 3a). However, at wavelengths>500 nm, the differences in optical responses are smaller, such that the photocatalytic activity under 540-nm irradiation is less pronounced. Nevertheless, the nitrogen-doped titanium dioxide nanocrystals still exhibited higher photocatalytic activity.

A second method of making doped titanium dioxide nanoparticles embodying features of the present invention includes oxidizing a powder having a formula TiX, wherein X is selected from the group consisting of boron, carbon, silicon, germanium, nitrogen, phosphorous, arsenic, sulfur, selenium, tellurium, fluorine, chlorine, bromine, iodine, and combinations thereof to form titanium dioxide nanoparticles doped with X. In some embodiments, X is selected from the group consisting of carbon, nitrogen, sulfur, and phosphorous.

In some embodiments, as further explained below, the oxidation-based approach described above for making doped titanium dioxide nanoparticles further includes heating the powder. In some embodiments, the heating temperature is incrementally increased during the heating process. In some embodiments, the heating temperature is increased at a rate of about 2° C. per minute. In some embodiments, at least a portion of the heating is performed at a temperature of between about 250° C. and about 700° C. In some embodiments, the oxidation of TiX is performed using ambient air. In other embodiments, the oxidation is performed using oxygen gas (e.g., under an oxygen atmosphere).

Typically, the concentration of dopant in nanocrystals formed with this method is higher in the core of the nanoparticle than in the shell, which renders these materials particularly suitable for use as photovoltaics. Moreover, it should be noted that the dopant concentration profile of nanoparticles prepared by the oxidation-based approach is the opposite of that for nanoparticles prepared by the hydrolysis-based approach. Thus, methods in accordance with the present invention provide the ability to prepare nanoparticles tailor-made for specific applications. By way of example, it may be desirable to prepare nanoparticles for use as catalysts by the hydrolysis-based approach since a high shell concentration of dopant is typically desirable in catalytic applications. Similarly, it may be desirable to prepare nanoparticles for use as photovoltaics by the oxidation-based approach since a high core concentration of dopant is typically desirable in photovoltaic applications.

An advantage of the oxidation-based method for preparing doped titanium oxide nanoparticles is that many of the titanium-containing starting materials (TiX) are commercially available (e.g., TiN, TiC, TiS₂, TiP, etc.), thereby simplifying the synthetic process.

In the oxidation-based approach for making doped titanium dioxide nanoparticles, the concentration of dopant may be varied by adjusting the temperature and duration of the oxidation.

Of the various methods that have been developed for synthesizing TiO₂ nanoparticles, many result in nanocrystals with a tendency to aggregate. However, it has been discovered that highly dispersed TiO₂ nanocrystals may be obtained by the simple oxidation-based method described above, which involves the controlled oxidation of commercially available micrometer sized TiX (e.g., X═C, N, S₂, P, etc.) powders under ambient conditions. Bulk titanium carbide, nitride and sulfide have metallic or semi-metallic properties, and may be oxidized into micrometer sized titanium dioxide in air or oxygen atmosphere as noted above. The TiO₂ nanocrystals thus prepared have long-tail absorption in the visible region, making them good candidates for visible-light applications.

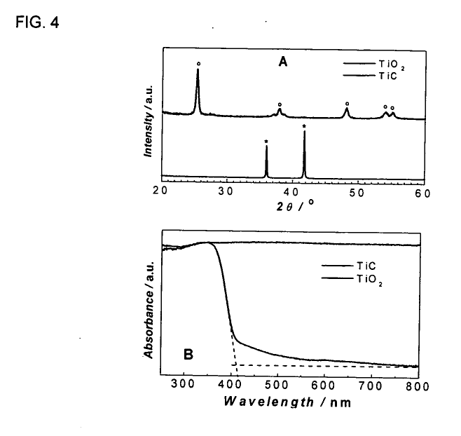

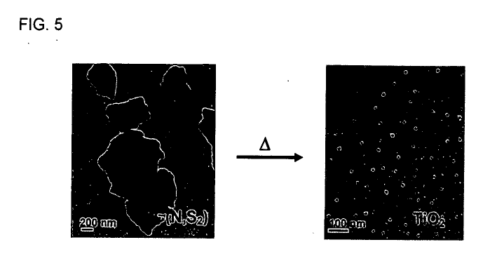

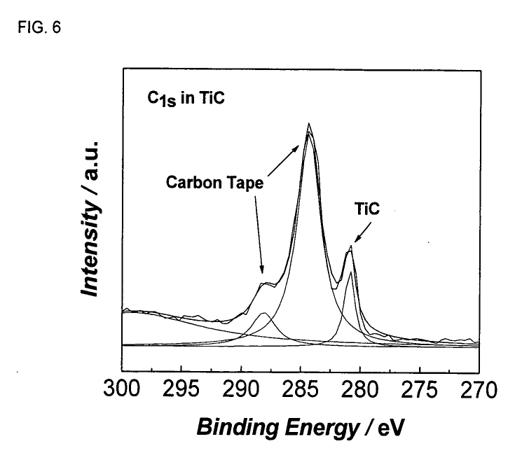

FIG. 4A shows the XRD pattern of commercial TiC powder before and after heating at 350° C. for 96 hours, obtained on a Philips PW 3710 X-ray powder diffractometer. Before heating, the TiC powder typically has a cubic structure and micrometer sizes. After heating, the micrometer sized TiC powder converted completely into the anatase TiO₂ structure, and the average size of the resultant powders was about 25 nm in diameter as calculated by the Debye-Scherrer's equation. FIG. 5 shows images of the micrometer sized TiC powder starting material and the TiO₂ nanocrystals that are formed after controlled oxidation. Complete oxidation of the titanium carbide after heating is supported by the XPS spectra shown in FIGS. 6-9, which were taken on a Perkin-Elmer PHI 5600 XPS System with the samples on the carbon tape sticking to the aluminum support. The carbon 1s signal from TiC at 480.9 eV disappeared, and the titanium 2p_{3/2} peak shifted from 454.3 eV to 458.6 eV, typical values for Ti 2p₃₁₂ binding energies in TiC and TiO₂ compounds, respectively.

As shown in FIG. 6, the C is core-level XPS pattern of commercial TiC powder contains the C Is peaks at 280.9 eV and peaks from the supporting carbon tape at 284.3 eV and 288.1 eV (satellite). The peaks were fitted with Lorentzian functions.

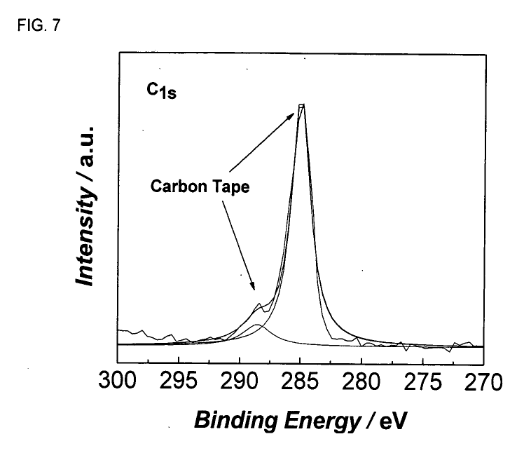

As shown in FIG. 7, the C 1s core-level XPS pattern of TiO₂ nanocrystals obtained by heating commercial TiC powder contains only the C 1s peaks from the supporting carbon tape at 284.6 eV and 288.6 eV (satellite). The peak from the TiC sample at 280.9 eV has completely disappeared. The peaks were fitted with Lorentzian functions.

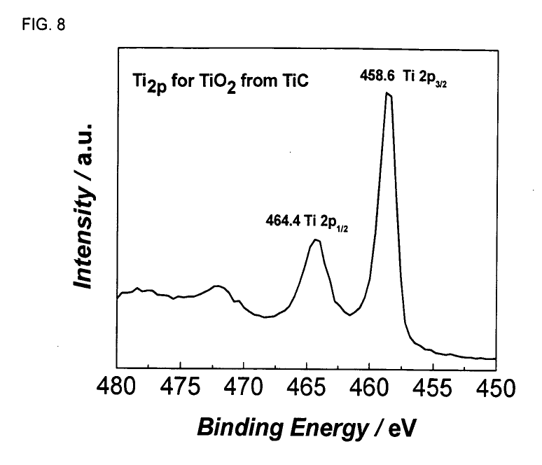

As shown in FIG. 8, the Ti 2p core-level XPS pattern of TiO₂ nanocrystals obtained by heating the commercial TiC powder displays only the Ti 2p peaks typical for TiO₂ at 458.6 eV (Ti 2p_{3/2}) and 464.4 eV (Ti 2p_{1/2}). This suggests that the TiC powder is completely transformed into TiO₂ after the heating.

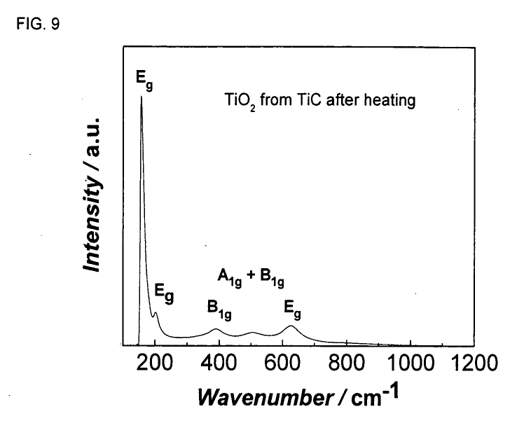

As shown in FIG. 9, the Raman spectrum of the TiO₂ nanocrystals obtained by heating TiC powder displays the typical vibrational modes of anatase TiO₂ patterns. There are five observed Raman active fundamental modes at 155 cm⁻¹ (E_{g}), 199 cm⁻¹ (E_{g}), 389 cm⁻¹ (B_{1g}), 505 cm⁻¹ (A_{1g}+B_{1g}) and 626 cm⁻¹ (E_{g}). The sample is excited at 647.0 nm.

Commercial TiC powder is black. However, its color changed after heating to light yellow. FIG. 4B shows the reflectance spectra of TiC power and the resultant TiO₂ nanocrystals after heating. Before heating, the TiC absorbed almost every wavelength in the visible regime. After heating, the resultant TiO₂ nanocrystals had a band-edge absorption around 3.0 eV (414 nm), and a defect-state absorption from 414 nm to 800 nm. The long wavelength absorption suggests that TiO₂ nanocrystals thus prepared are good candidates for visible-light photocatalytic applications.

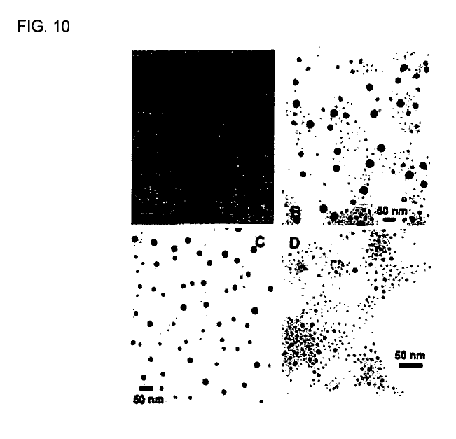

FIG. 10 shows transmission electron microscopy (TEM) images of the TiC powder before and after heating, which were obtained on a JEOL 1200EX transmission electron microscope operated at 80 kV. Samples for TEM were prepared by depositing a drop of a nanocrystal solution in water onto a copper grid supporting a thin film of amorphous carbon. The grid was dried in air. As shown in FIG. 10A, the size of the TiC powder was above 500 nm prior to heating. After heating, as shown in FIG. 10B, well-dispersed nanometer-sized TiO₂ particles were obtained in a mixture of sizes. The size distribution of these samples may be narrowed by a size selection process with a centrifuge. FIGS. 10C and 10D show two near monosized TiO₂ nanoparticles after the size selection process.

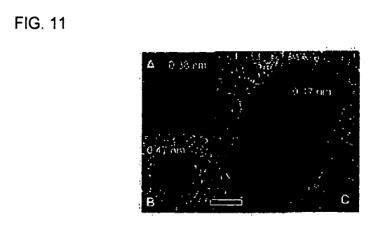

FIG. 11 shows HRTEM images of the resultant TiO₂ nanocrystals, which were obtained on a Tecnai F30 operated under 300 kV. The resultant TiO₂ nanoparticles were highly crystallized and the lattice structure may be resolved in these particles. Most TiO₂ nanoparticles had substantially spherical shapes and single domains of crystallization (FIGS. 11A and 11B), while some nanoparticles had irregular shapes and bigger sizes with several domains of crystallization (FIG. 11C). It is presently believed that the smaller nanocrystallites were formed from the disintegration of larger crystals during heating. The distance between the lattice planes of the TiO₂ nanocrystals was obtained as 0.47 nm for (002) and 0.38 nm for (100) planes.

In the embodiment described above, TiC is oxidized to provide C-doped titanium dioxide nanocrystals. In other embodiments, titanium dioxide nanocrystals containing different dopant atoms may be prepared analogously by oxidizing the appropriate TiX starting material.

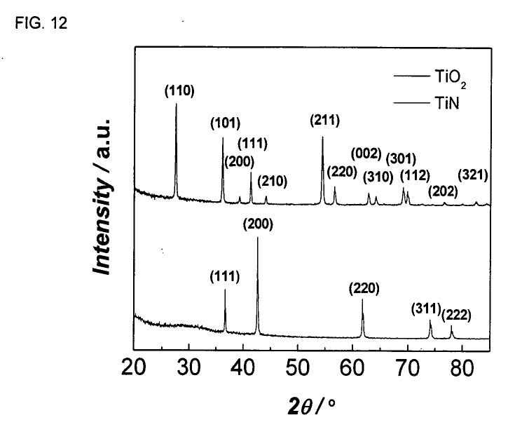

By way of example, TiN and TiS₂ powders also transformed into TiO₂ after direct heating under an ambient atmosphere. As shown in FIGS. 12-16, XRD and XPS displayed the complete transformations for these materials. The resultant TiO₂ powders also displayed long-tail absorption from TiC.

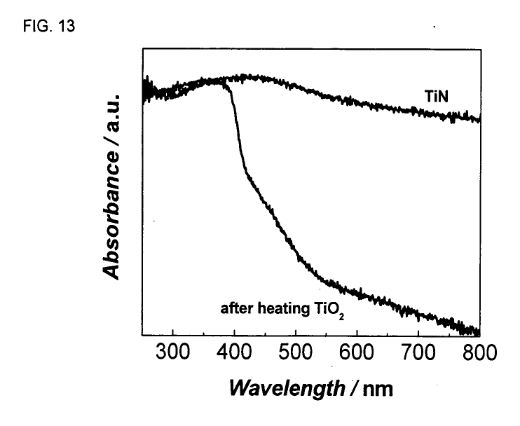

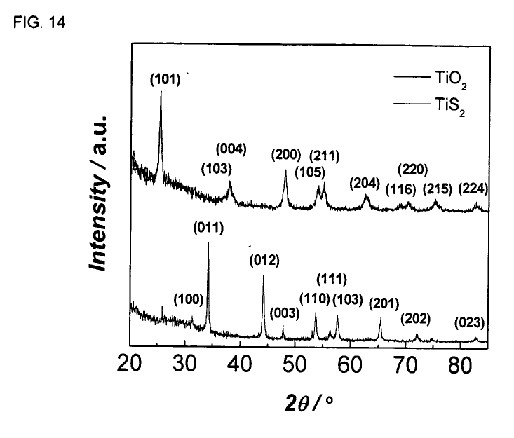

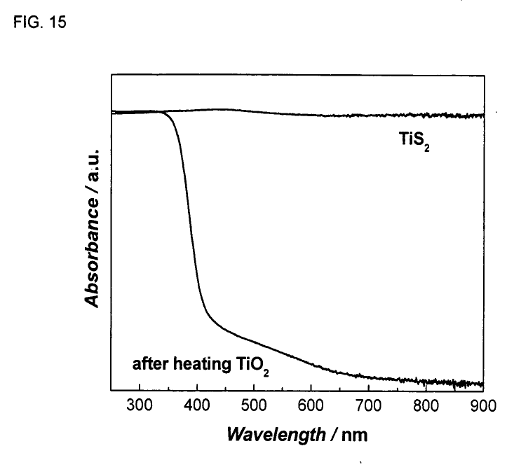

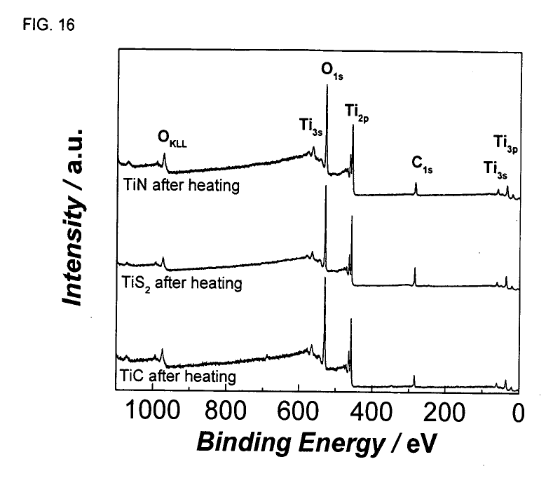

As shown in FIG. 12, the micrometer-sized TiN powder was completely converted into TiO₂ nanocrystals. Similarly, as shown by FIG. 14, the micrometer-sized TiS₂ powder was completely converted into nanometer-sized TiO₂ nanocrystals. Likewise, as shown in FIG. 16, each of TiC, TiN and TiS₂ were completely converted into TiO₂ after heating. The carbon Is signals at 284.3 eV in the spectra were from the carbon tape, which can be differentiated from the carbide signal (280-281 eV). In FIGS. 13 and 15, the visible absorption is displayed in the region of 400 nm to 800 nm.

The chemical reactions associated with the oxidation-based preparation of doped titanium dioxide nanoparticles described above may be summarized by the following equation:
TiX(X═C,N,S₂)+(1+m/2)O₂→TiO₂+XOₘ (1)

TiS₂ powder is easily transformed into anatase phase TiO₂ nanocrystals by procedures similar to those described above. The transformation temperature (450° C. for 96 hrs) is higher for TiS₂ than for TiC powder (350° C. for 96 hrs). In the case of TiN, the transformation temperature into TiO₂ is even higher than that for TiC powder (650° C. for 96 hrs), and the resultant powders are typically a mixture of anatase and rutile. Moreover, the size of the resultant nanocrystals is typically larger than that of nanocrystals made from TiC and TiS₂ powders. The difference in the transformation temperatures for converting TiX into TiO₂ is directly related to the Gibbs free energy change, and decreases in the order of TiC>TiS₂>TiN, where the gas formed with each is assumed to be CO₂, SO₂ and NO₂, respectively.

The electronic structure of the visible light-activated, doped titanium dioxide nanoparticles prepared from TiC, TiN and TiS₂ will now be described. The band structure of the resultant TiO₂ nanoparticles was studied by using x-ray absorption, emission, and photoemission spectra. A comparison of the constructed band structure with a calculation for single crystal TiO₂ shows the validity of the approach taken and provides an explanation for the origin of the visible-light absorption properties of the TiO₂ nanoparticles.

The electronic structure of titanium dioxide is characterized by a band gap located in between the oxygen 2p valence band and the titanium 3d conduction band. The interaction between the Ti 4sp states and the oxygen 2p states forms a completely filled bonding combination of the oxygen 2p band, and a completely empty antibonding combination of the metal 4s and 4p bands. The bonding between the Ti 3d states and the oxygen 2p states leads to antibonding that splits the 3d states into the t_{2g} and e_{g} manifolds.

TiN and TiC are metallic conductors with a partially filled band and a chemical bond of simultaneously metallic, covalent, and ionic character, while TiS₂ is regarded as a semiconductor or semimetal, with a bandgap of about 0.9 eV. As described above, the oxidation of these materials thus provides an attractive route for preparing three types of doped TiO₂ nanoparticles.

The valence band, conduction band, and core level states of the doped TiO₂ prepared by oxidation of TiN, TiC, and TiS₂ were measured using a combination of x-ray absorption spectroscopy (XAS), x-ray emission spectroscopy (XES), and x-ray photoelectron spectroscopy (XPS). XES and XAS are powerful probes for investigating the occupied and unoccupied, respectively, electron densities of states in materials. In XAS, a core-level electron absorbs an x-ray photon and is excited to an unoccupied state above the Fermi level, E_{F}. The transition is controlled by the dipole selection rule, such that only transitions with ΔI=±1 are allowed. In addition, due to the higher probability of intra-atomic transitions, XAS is site selective with on-site transitions dominating the spectrum. In XES, a valence band electron de-excites via the emission of an x-ray photon, filling a core hole created by the x-ray absorption process. The XES spectrum reflects the element specific valence band density of states (DOS), resolved into its orbital angular momentum components (i.e., the partial density of states or PDOS). By contrast, XPS measures the total DOS of the valence band as well as the binding energy of the core-level electrons.

Thus, the electronic properties of the TiO₂ samples obtained by oxidizing TiN, TiC, and TiS₂ at high temperature were explored with XPS, XAS, and XES. A comprehensive picture of the band structure is derived from these measurements, including a construction of the conduction band from the combined spectroscopies. The constructed band structure is in good agreement with the experimental and theoretical results, which facilitates understanding of the origin of the long-tail visible-light absorption of the resultant TiO₂. TiN and TiC have NaCl-like cubic structures, while TiS₂ has a Cdl₂-like layered structure. TiN and TiC crystallize in the rock-salt structure, with the N or C atoms occupying interstitial positions in a close packed arrangement of Ti atoms. This structure gives rise to strong metal to metal and metal to non-metal interactions. TiS₂ has a layered structure consisting of sulfur-titanium-sulfur slabs. The titanium ions are in a regular octahedral coordination to the sulfur ions and each slab is formed by a two-dimensional hexagonal titanium sublattice sandwiched by two closely adjacent sulfur hexagonal sublattices. The sulfur-titanium bonds are quite strong within a single slab, whereas there is only a weak coupling between individual slabs.

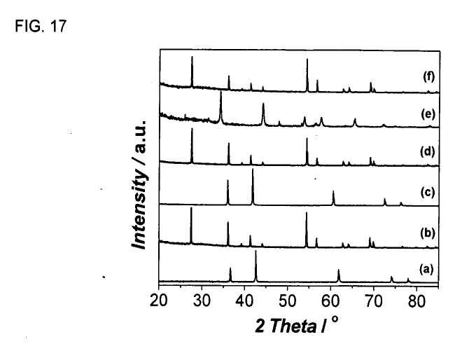

FIG. 17 shows the change in crystal structure of the starting materials TiN, TiC and TiS₂ into TiO₂ upon heat treatment. After heat treatment at 1000° C. in air, all of these materials completely oxidized into TiO₂ and transferred into rutile structures. FIG. 17 shows that the resultant samples obtained from TiN, TiC and TiS₂ display the diffraction pattern typical of rutile TiO₂.

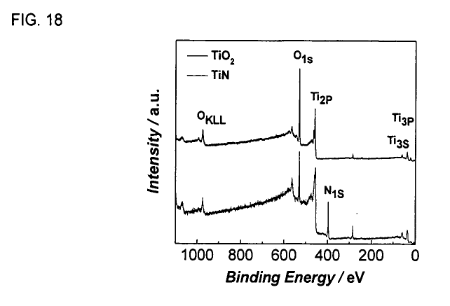

FIG. 18 shows wide energy range XPS spectra of TiN before and after heating. Before heating, some oxygen is detected, which suggests that the TiN samples were partially oxidized in air and/or some oxygen molecules were absorbed on the TiN surface. After heating, no nitrogen signal was detected, and the sample transformed completely into TiO₂, consistent with the XRD results. The resultant TiO₂ samples derived from TiN, TiC and TiS₂ display the typical bandgap around 3.1 eV of rutile phase TiO₂, and additional lower energy tail absorption.

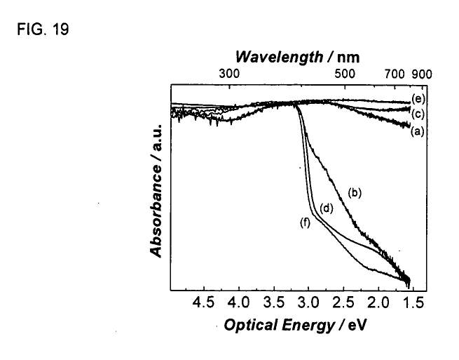

The chemical oxidation and structural transformation of TiN, TiC and TiS₂ directly affect the optical properties of these materials. FIG. 19 shows the optical reflectance of these materials before and after heating. TiN, TiC and TiS₂ have gray, black, and black-gray color. After heating, these samples changed into a yellow color or a slight yellow color (for TiS₂). As described above, TiN and TiC are good metallic conductors with a partially filled band at E_{F}, while TiS₂ is regarded as a semiconductor or semimetal with a bandgap of about 0.9 eV. In the optical spectrum, the original starting materials all show absorptions from the IR into UV regimes. After heating, they are oxidized into TiO₂, and display a bandgap around 3.1 eV due to the chemical and structural changes. Besides the bandgap absorption, these TiO₂ materials showed low energy absorption from the near-IR to the visible range. These changes may be attributed to defect formation during the structural transformation or residual impurities after heating. These optical changes can greatly improve the photocatalytic activity of doped TiO₂ compared to pure TiO₂, because the residual carbon, nitrogen or sulfur is a dopant.

For TiN, TiC, TiS₂ and TiO₂ compounds, the Ti 3d, 4s, and 4p atomic orbitals, and the nitrogen, carbon, and oxygen 2s and 2p or sulfur 3s and 3p atomic orbitals are involved in bonding. The valence-conduction band of the 3d metals consists primarily of mixed 3d, 4s and 4p states with the s states mainly responsible for electrical conductivity and cohesion, and the d states responsible for the majority of the fundamental transition-metal characteristics. To obtain information about the distribution of s and d states, the x-ray emission or absorption spectra arising from transitions to an inner level of p symmetry may be studied, such as the transition 3d4s →2p_{3/2} (the L₃ band or L_{α} line) and the transition 3d4s →2p_{1/2} (L₂ band or L_{β} line). The intensity distribution of a valence emission band and the variation in the absorption coefficient μ are directly related to the density of states and the transition probability given by the relation:
I(E), μ(E)˜P(E)·N(E) (2)

where P(E) is the transition probability and N(E) is the density of states. The energy distances between different characteristics of the emission and absorption spectra are equal to the corresponding distances on the density-of-state curve. For the 3d transition metals, the L_{2,3} absorption spectra give information regarding the unfilled band (conduction band), while the L_{2,3} emission band contains information about the filled portion of the 3d4s band (valence band). For the nonmetal, the K absorption or emission band of the oxygen in these compounds provides information regarding the evolution of the conduction-valence band from the contribution of TiO₂ during the above-described transformations.

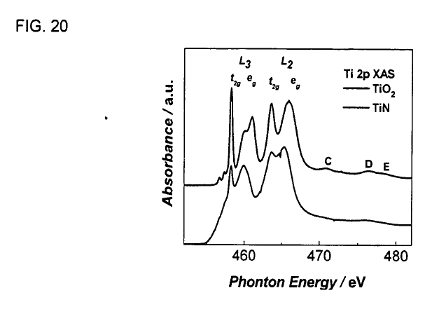

FIG. 20 shows x-ray absorption spectra for TiN and TiO₂ obtained by the total electron yield (TEY) method. In 3d transition metal oxides, the 2p x-ray absorption spectra are completely dominated by the strong 2p→3d dipole transitions. To a first approximation, the spectrum is related to the density of Ti 3d unoccupied states distorted by the influence of the Ti 2p core-hole potential. The features of the Ti L_{2,3}-edge spectrum reflect the local coordination number and symmetry of a titanium ion in the material and may be explained by the ligand-multiplet theory due to the large Coulombic and exchange interactions of the 2p-3d and 3d-3d orbitals. Thus, a direct relation to the local symmetry and electron configuration of both the ground state and the final state is possible.

The crystal field also splits the final state multiplet and the spectrum is dependent upon the crystal field effect of the final state. For both TiN and the resultant TiO₂, the 2p spin-orbit coupling splits the initial state into 2p_{3/2}, and 2p_{1/2}, resulting in two L-edge features denoted L₃, and L₂, respectively. Both the L₃ and L₂ features further split into L₃-t_{2g}, L₃-e_{g}, L₂-t_{2g}, and L₂-e_{g} features because of the low symmetry of the ligand field Oₕ. For TiO₂, the L₂-e_{g} feature splits further into a double peak band centered at 461 eV due to the slight distortion of the TiO₆⁸⁻ octahedron in rutile, which results from the configurational deformation into D2h predicted by the Jahn-Teller theorem. For TiN, this splitting does not occur since the TiN₆⁸⁻ forms a perfect Oₕ symmetry.

The excessive e_{g} broadening in the TiN spectrum is related to solid-state broadening and the Frank-Condon effect. The energy splitting for the centers of t_{2g}-e_{g} is 3.0 eV in TiO₂ and 2.5 eV in TiN. The difference is primarily due to the splitting of the e_{g} band in TiO₂. The first two small peaks are related to the splitting of a single allowed j-j coupling transition by the crystal field. There are three final states, which in order of increasing energy are: 2p_{3/2}3d_{3/2}, 2_{3/2}3d_{5/2}, and 2p_{1/2}3d_{3/2}. The 2p_{3/2}3d_{3/2} state gives rise to the two small peaks in the spectrum through the crystal field splitting. These two small peaks are also assigned to the subbands of the L₃-t_{2g} band by the crystal field splitting. The third and fourth peaks are related to t_{2g} and e_{g} symmetry for the L₃ edge. In the photon energy range 459-462 eV, the peak is attributed to e_{g}. The e_{g} peak at the lower energy side originates from the long Ti—O bonds due to a hybridization effect weaker than the short Ti—O bonds. The same consideration is used for the fifth and sixth peaks for the L₂ edge. Details of the L₂ edge are blurred because the intrinsic broadening is considerably larger due to an extra Auger decay channel.

The decrease in intensity ratio of the titanium 3d band to the 4sp band in titanium dioxide as compared to titanium nitride may be explained by the decreased hybridization of the titanium 3d orbitals with the oxygen 2p orbitals as compared to the hybridization with the nitrogen 2p orbitals in titanium nitride. Above 469 eV, there are weak and broad satellite peaks for both TiO₂ and TiN. The O 2p and Ti 2p states are the main contributors to the peak C in the satellite, without the titanium 3d and 4s states. Peak D is from O 2p and titanium 4p states with comparable contribution from Ti 3d states. Peak E is attributed to the titanium 4s and 4p states with the O 2p contribution. The satellite intensity depends strongly on the Coulomb attraction between the core hole and the 3d electrons U_{dc}, and the Coulomb repulsion between the 3d electrons U_{dd}. With increasing U_{dc}, the ligand electrons may easily be transferred to the 3d states, leading to increased satellite intensities. With increasing U_{dd}, the satellite intensity decreases. This suggests that U_{dc} is larger and U_{dd} is smaller in the resultant TiO₂ than in TiN. In addition, the spectrum of the resultant TiO₂ shows sharper structures which are characteristic of transition-metal oxides with large intra-atomic interactions. This suggests that the 3d electron and 4sp electrons of Ti are more delocalized in the TiN materials, which partially explains the good conductivity of TiN.

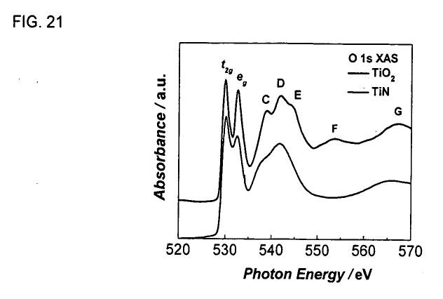

The O 1s absorption spectra are presented in FIG. 21. The O 1s x-ray absorption spectrum corresponds to the transition from the oxygen 1s core level into empty or partially filled O 2p states. The final state of oxygen 1s x-ray absorption contains a core hole in the oxygen Is level. At the absorption edge, the excited electron is then located in the Ti 3d band, and according to dipole selection rules has oxygen p character. Thus, the oxygen 1s x-ray-absorption spectrum gives a direct picture of the oxygen p-projected density of states.

The intensity and the spread of the absorption signal illustrate the importance of the covalent contribution to bonding in the material. The low-energy range of the spectra (528-535 eV) is dominated by two strong broad bands with an energy splitting close to the energy difference of peaks (i.e., to the splitting of t_{2g} and e_{g} states in the Ti 2p XAS). This region is attributed to the oxygen 2p contribution to states with predominantly titanium character (the titanium 3d band), or the O 2p states which are hybridized with the empty Ti 3d bands. Crystal field effects split the spectrum.

The prominent doublets may be assigned to transitions into the t_{2g} and e_{g} bands of the titanium, respectively, and are sensitive to the short-range order (i.e., the nearest neighbor order) and are more localized. For the resultant TiO₂, the t_{2g} peak is centered at 529.9 eV and the e_{g} peak is centered at 532.6 eV. For TiN, the t_{2g} peak is centered at 530.1 eV and the e_{g} peak is centered at 532.5 eV. The energy splitting of these two peaks is larger (2.7 eV) in the resultant TiO₂ than in the TiN sample (2.4 eV). The crystal field splitting (10 Dq) is very sensitive to coordination number, the distribution of the ligand, and the strength of the hybridization. This region presents only weak dispersion effects and reflects short-range-order interaction.

That the peaks are weaker and broader in TiN samples than in the TiO₂ sample suggests that the oxygen atom in TiN has some disorder in relation to the Ti atom. The high-energy part of the spectrum is formed by the delocalized states derived from the antibonding O 2p and Ti 4sp band with principally oxygen 2p character. There is a large degree of hybridization between the Ti excited 3d and O 2p valence band. The features above 536 eV are due to the covalent mixing of O 2p and Ti 4sp orbitals and are sensitive to the long-range order. This region presents larger dispersion effects. In this region, the difference between TiN and TiO₂ of the O 2p coupled with Ti 4sp suggests the lack of long-range order in the thermal oxide layer. In the low energy region, the t_{2g}-e_{g} splitting is slightly smaller in TiN than in TiO₂. This is attributed to the weaker Ti 3d-O 2p interactions caused by the presence of O vacancies and other defects, as well as the significant deviation from the TiO₂ stoichiometry. This is also verified by the stronger signal in the satellite region in TiO₂ than in TiN.

In the case of TiN, the splitting and the width correspond to the ideal ligand coordination. Lattice distortion and point defects affect the ligand distribution and cause broader t_{2g} and e_{g} bands. Defects such as vacancies lower the coordination number and results in smaller splitting. The origin for the bands C-E is the same as described above in the Ti XAS spectra. Peaks F and G are attributed to O 2p with contribution from Ti 4p. The weaker peaks for TiN suggest that the oxygen has not completely replaced the nitrogen and coordinated with the titanium atoms in the lattice. The states in the 10 eV to 20 eV region above the threshold of the absorption are dominated by oxygen p character and they are the antibonding combinations of direct oxygen-oxygen interactions. The stronger signals of TiO₂ compared to those of TiN suggest stronger oxygen-oxygen interactions in TiO₂ than in TiN, or more organized oxygen in TiO₂ as compared to disordered oxygen in TiN.

X-ray emission bands result from removal of an inner-level electron followed by the transition of a valence-band electron into the inner hole. Such transitions give rise to the K band (valence band→1s), the L_{2,3} bands (valence band→2p), and the M_{2,3} bands (valence band→3p). The K band results from probing the part of the valence band having p symmetry while the L band results form the distribution of s and d symmetry. The band structures of TiN, TiC, TiS₂ and TiO₂ are composed of a strong admixture of p, d, and s symmetry, so that the information present in the K and L bands may be combined to explore the valence band structure.

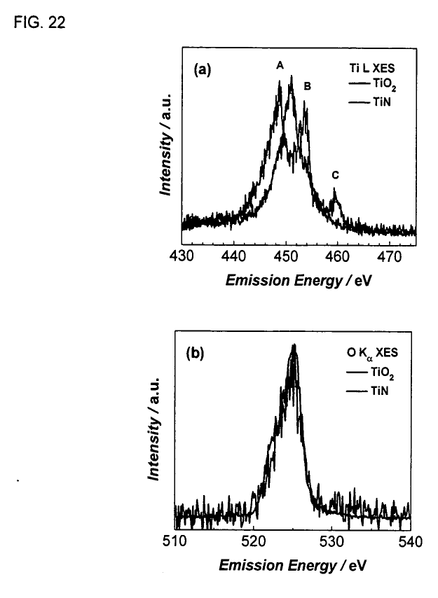

FIG. 22 shows the XES spectra of Ti L and O K_{α} in TiN and the resultant TiO₂ samples. The valence band of TiN and TiO₂ is mainly composed of the contribution from the major nitrogen or oxygen 2p states with minor contribution from the titanium 3d states. The titanium L emission spectrum of TiN shows three emission bands. Band A (448.2 eV) represents the transition from the nitrogen 2p band mixed with the Ti 3d to titanium 2p_{3/2} band. Band B (453.5 eV) is a combination of titanium 3d to titanium 2p_{1/2} transitions. Band C (459.7 eV) is due to the transition form Ti 3d to Ti 2p_{1/2}. The main peak at 450.6 eV in the resultant TiO₂ represents the transition from the oxygen 2p band to the Ti 2p_{3/2} band, and the small shoulder at the higher energy side (454.6 eV) is due to the overlap with the transition from the oxygen 2p band to the titanium 2p_{1/2} band.

The O K_{α} x-ray emission spectra of both TiN and the resultant TiO₂ show two peaks, with a main peak at 525.0 eV for TiO₂ and at 524.9 eV for TiN, and a smaller shoulder at 523.1 eV for TiO₂ and 522.6 eV for TiN. The O K emission may be attributed to the transition from the filled oxygen 2p states to the oxygen 1s hole state. The splitting between the main peak and the shoulder may be attributed to the crystal field splitting or the different oxygen state in the sample. The higher oxygen K_{α} emission energies in the resultant TiO₂ compared to TiN suggest that the energy difference between the O 1s core-level and the O 2p states in the valence band is larger in TiO₂ than in TiN, due to their different chemical compositions, crystal structures and symmetries.

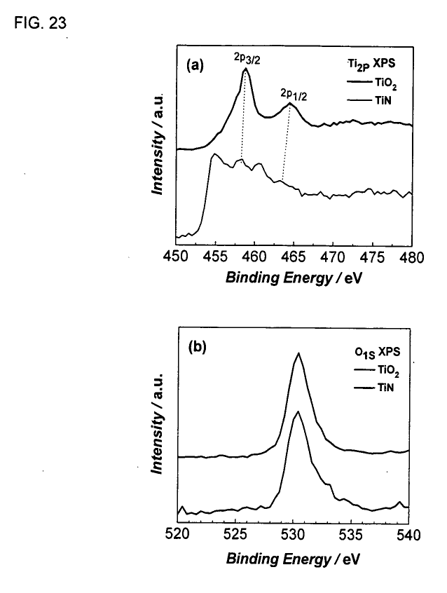

In the XPS measurement, the electrons were promoted to the vacuum state and may be considered as free electrons. Thus, the outgoing electrons directly measure the binding energy of the core level, which may be used to determine the chemical states of different elements. FIG. 23 shows the Ti 2p core-level and O 1s core-level photoemission spectra for TiN and the TiO₂ obtained after heating. In the titanium photoemission spectrum for TiN, there are four peaks with two (454.8 eV for 2p_{3/2} and 460.5 eV for 2p_{1/2}) from the TiN and two (458.1 eV for 2p_{3/2} and 463.3 eV for 2p_{1/2}) from TiO₂ that was formed after TiN was exposed to atmosphere. For the TiO₂ sample, there are two peaks centered at 458.7 eV and 464.4 eV with an energy difference of 5.7 eV. These are assigned to the 2p_{3/2} and 2p_{1/2} core states of titanium with the chemical state 4+. The oxygen 1s core-level photoemission spectrum for the TiN sample may be fit by two overlapping Lorentzian functions giving a primary peak at 530.3 eV and a secondary peak at 532.0 eV. However, the O 1s spectrum from the resultant TiO₂ sample displays only one peak centered at 530.5 eV. The peak around 530 eV in both samples may be attributed to the oxygen coordinated to the titanium atom which has a local TiO₂ structure, while the peak at 532.0 eV in the TiN sample may be attributed to absorbed oxygen. From the ratio of these two peaks in the TiN sample, it may be concluded that the oxygen in the TiN mainly exists in the form of oxide.

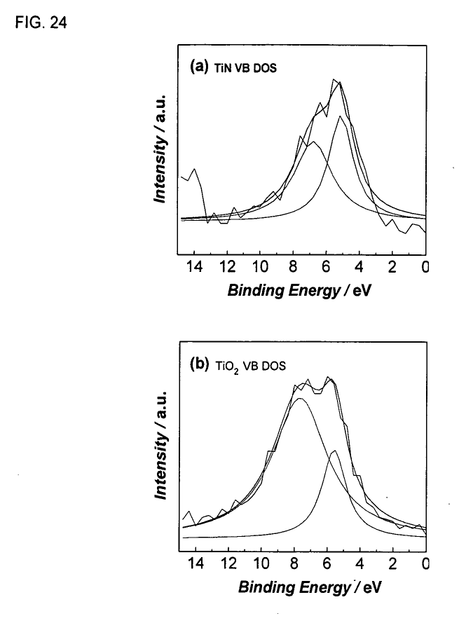

In contrast to the XAS and XES, which probe the partial density of the conduction/valence band, XPS valence band probes the total density of the states distribution in the valence band. FIG. 24 shows the valence band XPS spectra of TiN and the resultant TiO₂ obtained after heating. Fitting the spectra to Lorentzians shows that TiN has a main peak at 5.1 eV and a shoulder at 6.8 eV, while TiO₂ shows a major peak at 7.7 eV and a minor peak at 5.6 eV. The energy position of these two peaks may indicate the location of two main peaks of Ti 3d states in the valence band. The main part of the valence band of TiO₂ shifted to higher binding energy than that of TiN from the XPS core level spectra described above. The valence band XPS results suggest that the valence states have moved further away from the vacuum state, assumed to be at the zero binding energy. This may partly explain the wider bandgap of TiO₂ as compared to TiN.

Since the Ti 3d states contribute to the valence band, when the Ti 2p core electron is excited enough above the continuum state and the excited electron spreads out of the excited core site, a Ti 3d →2p emission spectrum is obtained. The Ti L emission corresponds to the transition from the Ti 2p core hole state to the valence hole state, each of which corresponds to the final state of Ti 2p and valence-band photoemission, respectively. Therefore, it is useful to relate the x-ray emission spectra with Ti 2p core and valence photoemission spectra, which may help elucidate the electronic structure of the material.

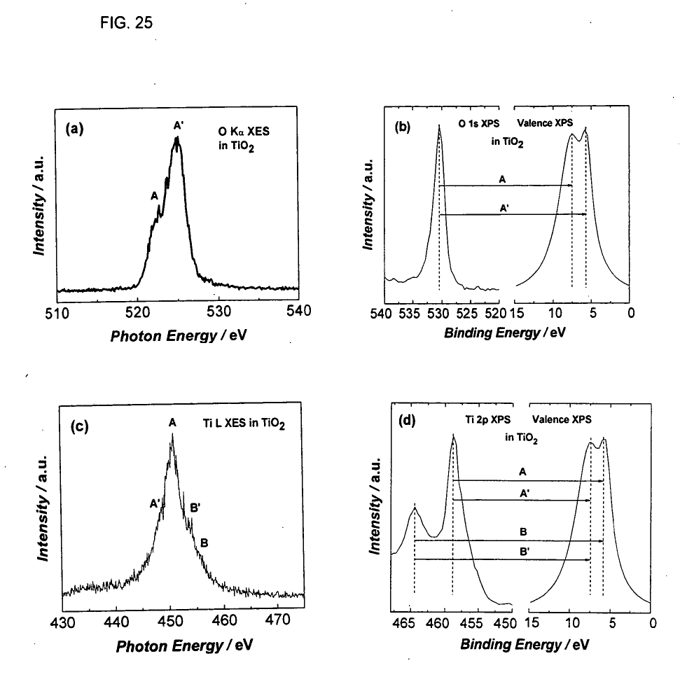

FIG. 25 shows the assignment of the O K_{α} x-ray emission spectrum (a) from the transitions between the O 1s core-level and the valence band structure (fitting curve) from XPS measurement (b). The peaks A and A′ are assigned to the transitions from the valence density of state levels to the O 1s core level. Although the two bands in the valence band almost show the density of states as determined from the XPS, the transitions to the O 1s core level show one main peak and one shoulder peak. This suggests that the transition probability to the same O 1s core level from the different states in the valence band is different. Since the states in the valence band are strongly hybridized, it is plausible that the transition probability varies. Similarly, the peaks in the Ti L x-ray emission spectrum may be assigned to transitions from the valence band levels to the Ti 2p core level (c) and (d), and the transition probability to the same Ti 2p core level is different from the different states in the valence band containing different O 2p and Ti 3d character. Given the ΔI=+1 selection rule, the features in the O K_{α} and Ti L emission spectra primarily reflect transitions from valence band states having O 2p character, and states having Ti 3d or 4s character, respectively.

Once the structure of the O 1s x-ray emission spectrum in TiO₂ is determined by consideration of the transitions from the measured core-level and valence-band states, it should be possible to retrieve/construct the structure of the valence band if the O K_{α} x-ray emission spectrum and the O 1s XPS spectrum are known. From the XPS valence band photoemission, the total density of states is obtained, while from the x-ray emission, only the states involving a high transition probability and having the same character (i.e., O 2p) may be obtained. It is reasonable to assume that the transition probabilities from the different valence density of states having the same character (i.e., O 2p) to the O 1s core level are the same. Thus, the partial valence band structure having O 2p character may be constructed.

In the reconstruction of the conduction band, the object is to relate the x-ray absorption features with the core-level XPS spectrum. In XPS, electrons are excited to vacuum state (zero binding energy), while in the x-ray absorption spectra, the electrons are assumed to be excited to the conduction band. Thus, the energy difference of the corresponding XPS spectra and the x-ray absorption spectra (the core-level binding energy minus the x-ray absorption peak energy) gives the energy distribution of the states in the conduction band. If the core-level binding energy is larger than the x-ray absorption peak energy for the same O 1s, or Ti 2p electrons, the corresponding binding energy is a positive value. Otherwise, it has negative values. The other parameters of the peak, such as the width and relative height, are obtained from fitting the corresponding x-ray absorption spectrum. The constructed partial valence band (VB) structure having O 2p character from O 1s core-level XPS and O K_{α} x-ray emission spectra may be constructed as well as the partial valence band having Ti 3d character.

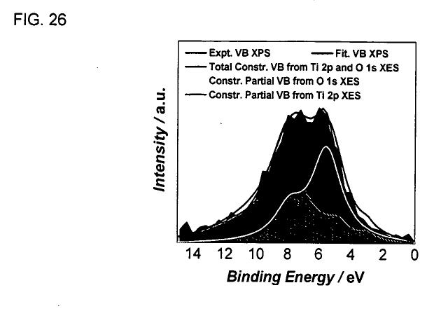

FIG. 26 shows a comparison of the sum of the partial VBs of O and Ti to the total valence band from XPS measurement with its fit. The total density of states as the sum of the partial VBs of the O and Ti matches quite well with the VB from the XPS measurement. The comparison of the reconstructed partial valence bands to the valence band from XPS measurement (with its fit) displays the validity of this method, where the partial VBs from O and Ti are contained in the total VB in TiO₂. Thus, from the core-level x-ray emission and XPS results of the components, the partial and total valence band structures may be retrieved, which match with the experimental measurement from XPS.

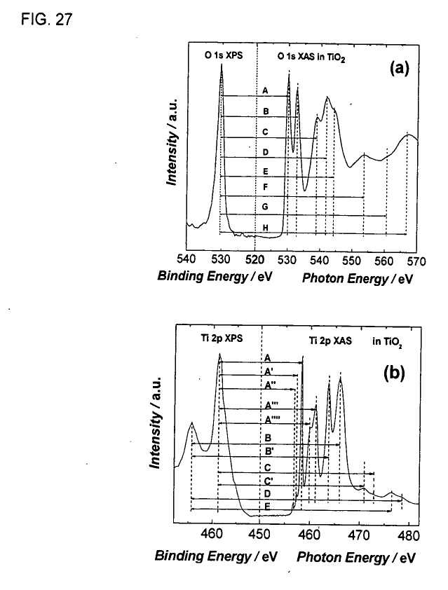

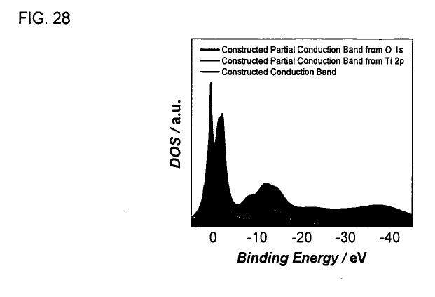

Using a similar approach of correlating XPS and XAS, as shown in FIG. 27, the O and Ti partial conduction bands may be constructed from the O 1s or Ti 2p core-level XPS and O 1s or Ti 2p x-ray absorption spectra, as shown in FIG. 28. The former contains mainly the component having or coupling with states of O 2p character, and the latter contains mainly the Ti 3d component in the conduction band.

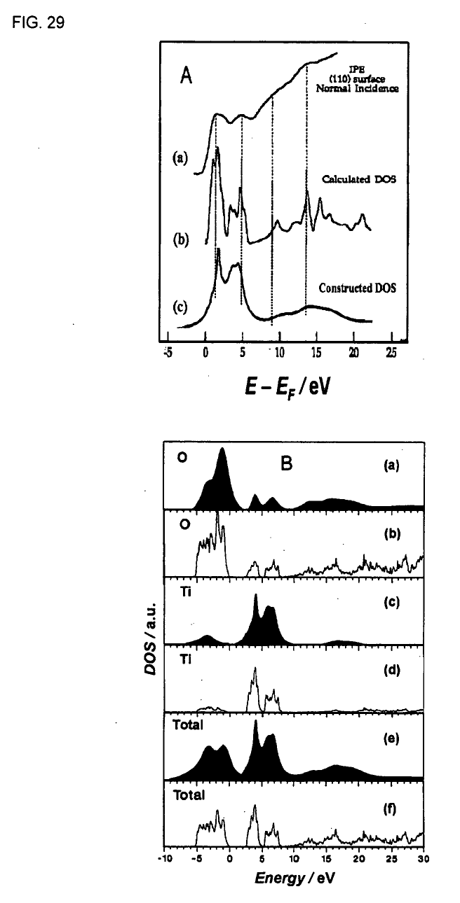

The sum of the O and Ti partial conduction bands should also reflect the total conduction band structure of TiO₂. FIG. 29 shows the constructed conduction band in the TiO₂ obtained from TiN. In FIG. 29, (a), (c), (e) correspond to constructed band structures and (b), (d), (e) correspond to theoretical calculations The comparison between the constructed conduction band was consistent with the inverse-photoemission spectrum, the calculation of the band structure, as shown in FIG. 29A, and the results from previous Bremstrahlung isochromat studies on TiO₂.

The constructed band structure may also be compared to the theoretical calculations in the literature, which are well established and quite accurate. FIG. 29B compares the constructed band structures of TiO₂ obtained from TiN in this contribution to a single particle calculation of the band structures in TiO₂. In the comparison, the constructed bands are shifted by the same value to match the positions of the t_{2g} and e_{g} peaks in the O partial band structures. The good match evident from FIG. 29 again displays the validity of the method to build band structure from XPS and x-ray absorption/emission spectra.

The difference between the constructed band structure and the theoretical calculation is that in the constructed band structure, there exist states in the region from 2.5 eV to zero in the valence band, which arise primarily from the anion. This difference is caused by and explains the difference between the samples and the calculation. In the calculation, pure rutile single crystal structure was used, and in the region below the bandgap (2.5 eV) there is no absorption. In the tests, the TiO₂ sample has long tail absorption from the visible into the near-infrared region in the reflectance measurement described above.

The additional states in the valence band may be attributed to residual anion from the precursor after heating, which is beyond the detection sensitivity of XPS. These additional states are consistent with the results that additional states appeared at the edge of the valence band from the calculation by Asahi (Science, 2001, 293, 269), where the full-potential linearized augmented plane wave formalism in the framework of the local density approximation (LDA) for C, N, F, P, or S doped TiO₂ crystal was used. This likely explains the origin of the long-tail absorption.

Thus, as described above, titanium nitride, titanium carbide, and titanium sulfide may be successfully transformed into titanium dioxide with rutile phase by treatment at high temperature. Compared to the normal near-UV absorption property of rutile titanium dioxide single crystal, the resultant titanium dioxide has long-tail absorption from the near-infrared through the visible to the UV region.

Methods of using and applying doped metal oxide nanoparticles in accordance with the present invention will now be described. It is to be understood that the specific doped metal oxide nanoparticles described below in connection with various experiments (e.g., N-doped titanium dioxide nanoparticles) are merely representative examples of other doped metal oxide nanoparticles embodying features of the present invention. Thus, it is to be understood the applications described below may utilize different metal oxides than titanium dioxide and different dopant atoms than nitrogen.

A method of treating an environmental contaminant embodying features of the present invention includes (a) providing one or a plurality of titanium dioxide nanoparticles containing a non-metallic dopant selected from the group consisting of boron, carbon, silicon, germanium, nitrogen, phosphorous, arsenic, sulfur, selenium, tellurium, fluorine, chlorine, bromine, iodine, and combinations thereof; (b) activating at least a portion of the nanoparticles with visible light to form activated nanoparticles; and (c) oxidizing at least a portion of the environmental contaminant by reaction with the activated nanoparticles (e.g., the activated nanoparticles may react directly with the environmental contaminant and/or catalyze the oxidation of the environmental contaminant using ambient air or other source of oxygen). In some embodiments, the environmental contaminant to be treated is a contaminant in waste water. In some embodiments, the environmental contaminant is a contaminant in fresh water. In some embodiments, the environmental contaminant is oil (e.g., seawater-soluble crude oil fractions).

The problem of crude oil pollution in the coastal zone has been mainly associated with tanker accidents. However, the major petroleum contamination source is actually related to human activities, including but not limited to shipping operations, refinery wastewater, and accidental spills. Wastewater is usually rich in dissolved organic carbon (DOC) from crude oils, which is referred to as the soluble crude oil fraction. This continuous load of organic waste in coastal waters is the main source of toxicity in coastal ecosystems. Biological treatments and activated carbon have previously been employed for removing these soluble organic impurities from water. However, these end-of-the-pipe controls are limited in their application. Hence, doped metal oxide nanoparticles embodying features of the present invention may be used to efficiently accelerate the oxidation and destruction of a wide range of organic contaminants in polluted waters.

A representative application in accordance with the present invention, wherein nitrogen-doped TiO₂ nanocrystals are used for the photocatalytic degradation of the three azo dyes shown in Table 1—acid orange 7 (AO7), reactive black 5 (RB5), and procion red MX-5B (MX-5B)—is described below.

TABLE 1
Characteristics of Acid Orange 7, Reactive Black 5, and Procion
Red MX-5B

| | | | Absorbance |
|---|---|---|---|
| Dye | Chemical formula | M_{w} (g/mol) | (λ) |
| Acid Orange 7 | 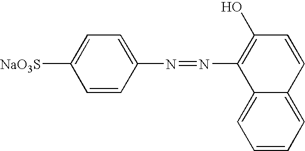 | 350.3 | 483 nm |
| Reactive Black 5 | 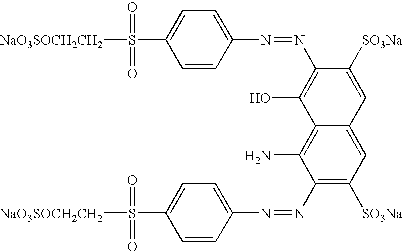 | 927.9 | 597 nm |
| Procion Red MX- 5B | 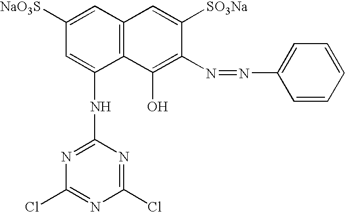 | 595.4 | 538 nm |

The anionic-doped titania nanocatalysts embodying features of the present invention demonstrated high reactivity under visible light (wavelength>390 nm), allowing more efficient usage of solar light. Experiments were conducted to compare the photocatalytic activities of nitrogen-doped TiO₂ nanocatalysts and commercially available Degussa P25 powder using both UV illumination and solar light. Nitrogen-doped TiO₂ after calcination had the highest photocatalytic activity of three catalysts tested, and decolorized 95% of AO7 in one hour under UV illumination. The doped TiO₂ also exhibited substantial photocatalytic activity under direct sunlight irradiation, with 70% of the dye color being removed in one hour and complete decolorization occurring within three hours. Degussa P25 did not cause detectable dye decolorization under identical experimental conditions using solar light. The decrease in total organic carbon (TOC) and evolution of inorganic sulfate (SO₄²⁻) ions in dye solutions were measured to monitor the dye mineralization process.

Azo compounds are an important class of synthetic dyes commonly used as coloring agents in the textile, paint, ink, plastics, and cosmetics industries. These compounds are characterized by the presence of one or more azo groups (—N═N—) bound to aromatic rings. The release of azo dyes into the environment is highly undesirable due in part to their coloration of natural waters, toxicity, and mutagenicity, and the carcinogenicity of the biotransformation products thereof. Azo groups, particularly the accumulation of azo, halogen, sulfo or nitro groups, confer xenobiotic character on synthetic compounds. Moreover, the electron-withdrawing character of these substituents generates an electron deficiency, rendering these compounds less susceptible to oxidative catabolism. For these reasons, conventional wastewater treatment plants, which rely on sorption and aerobic biodegradation, have negligible removal efficiencies for azo dyes. However, doped TiO₂-based nanoparticles embodying features of the present invention demonstrated enhanced photocatalytic efficiency when used with electron deficient model compounds, such as methylene blue in water. Therefore, the application of these materials for the degradation of industrial dyes, as further described below, represents a significant advance in the establishment of semiconductor-based photocatalysts for environmental use.

The process of semiconductor based photocatalysis may be represented as follows:
S+hν→e⁻_{CB}+h⁺_{VB} (3)

where S is a semiconductor material. Upon absorption of light energy equal to or larger than the band gap energy, a valence band electron of the semiconductor may be excited to the conduction band, leaving a positive hole in the valence band. The positive hole is a strong oxidant, which can either oxidize a compound directly or react with electron donors like water or hydroxide ions to form hydroxyl radicals (.OH), which are also potent oxidizers.

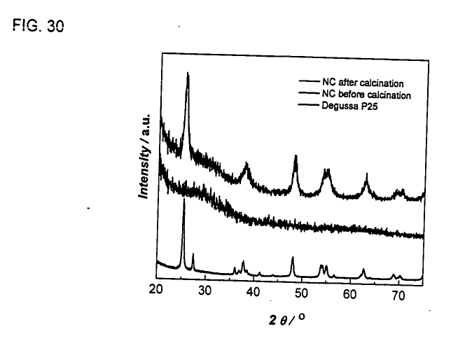

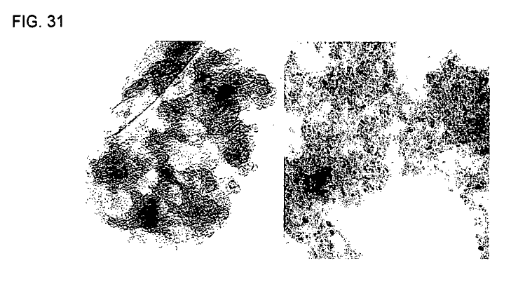

FIG. 30 shows the XRD patterns of the samples used in the above-described photodegradation studies. From the intensity distribution of the reflections and the integral intensity of the X-ray diffraction, the average nanocrystallite sizes may be calculated according to the Debye-Scherer equation. With diminishing nanocrystal size, the measured XRD pattern exhibits broadened peaks. The Degussa P25 powder consists of a mixture of anatase and rutile phases in the ratio of 3:1, while the nitrogen-doped TiO₂₋ₓNₓ nanoparticles, before and after calcinations, display only the anatase crystal phase, which is generally considered more photoreactive. Compared to the 30 nm average diameter of a commercial Degussa P25 sample, the average grain size of the N-doped TiO₂ is close to 10 nm, as estimated from the Debye-Scherer equation after calcination. The diffraction peaks of the nano-sized TiO₂ are broad since some peaks coalesce due to the small size of these nanoparticles. The X-ray patterns clearly show that before calcination, the doped TiO₂ nanomaterial is completely amorphous, while after calcination it is well crystallized. This is consistent with the result from TEM measurement. FIG. 31 shows TEM images for the doped TiO₂ nanocatalyst before (left) and after (right) calcination. Before calcination, the doped TiO₂ nanomaterial exhibits a gel-type and amorphous structure. After calcination, the sample crystallized with the crystallite size centered around 10 nm.

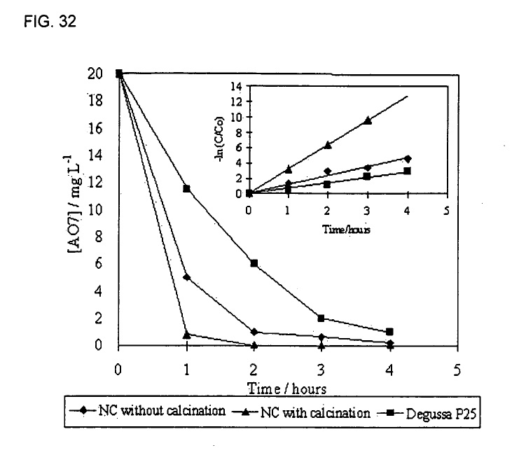

Doped TiO₂ nanomaterials and Degussa P25 were used to degrade AO7 using a UV light source. The reactions were carried out with solutions containing 0.06 mM AO7 and 10 mg L⁻¹ catalysts. FIG. 32 shows the relationship between dye concentration and irradiation time for each catalyst during the photocatalytic degradation of AO7. N-doped TiO₂ nanoparticles after calcinations exhibited the best decolorization efficiency with 95% of AO7 decolorized in 1 hour, while N-doped TiO₂ without calcination showed 75% conversion and Degussa P25 showed less than 40% conversion in the same test period. The kinetics of AO7 decolorization is presented in FIG. 32 by plotting the logarithm of the normalized dye concentration against irradiation time. Fairly good linear relationships were observed, indicating all reactions followed apparent first-order kinetics. The decrease of dye concentration with time may be rationalized by a modified Langmuir-Hinshelwood (L-H) mechanism:

$$- \frac{ⅆ C}{ⅆ t} = \frac{{k}_{r} {K}_{e} C}{1 + {k}_{e} C} \qquad ( 4 )$$

where C is the dye concentration, kᵣ is the apparent reaction rate constant, and Kₑ is the apparent equilibrium constant for the adsorption of the dye on the nanocatalyst surface. The integrated form of Eq. (4) is:

$$t = \frac{1}{{K}_{e} {k}_{r}} ln \frac{{C}_{0}}{C} + \frac{1}{{k}_{r}} ( {C}_{0} - C ) \qquad ( 5 )$$

When the concentration of dye is sufficiently low, equation (5) may be expressed as:

$$ln \frac{{C}_{0}}{C} = {k}_{r} {K}_{e} t = {k}^{′} t \qquad ( 6 )$$

The overall rate constants for AO7 decolorization in reciprocal hours are given in the following order: k′_{calcinated NCs} (3.22 hr⁻¹)>k′_{uncalcinated NCs} (1.20 hr¹)>k′_{P25} (0.73 hr⁻¹), indicating faster dye decolorization and higher catalytic activity for N-doped TiO₂ by a factor of approximately 4. The better performance of N-doped nanoparticles may be explained by the fact that the nitrogen doped nanoparticles have visible light absorption and smaller sizes. The enhanced photocatalytic activity of the calcinated sample compared to the uncalcinated sample may be explained by the fact that after calcination, the sample was better crystallized. The sample is amorphous before calcinations; after absorbing photons, the electrons and holes created may be deactivated in the catalyst itself before they can migrate to the surface and undergo photocatalytic reactions.

Experiments directed to the degradation of AO7, MX-5B and RB5 by solar light and titanium dioxide were carried out using the three different titanium dioxide nanocatalysts under direct sunlight irradiation, with an initial dye concentration of 0.03 mM and a catalyst loading of 10 mg L⁻¹. After 8 hours of illumination, no detectable decolorization was observed for any of the three dyes with P25-assisted photocatalysis. By contrast, nitrogen-doped TiO₂ nanostructures exhibited substantial photocatalytic activity within the time frame of the tests. The decolorization of AO7, MX-5B and RB5 using N-doped TiO₂ nanocatalysts with and without calcination is described below. The total organic carbon, final mineralization products of the three investigated azo dyes, and the effect of catalyst loading are also described.

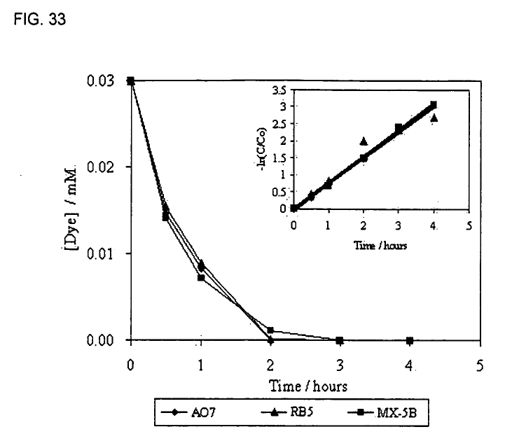

Three dye solutions were prepared with AO7, MX-5B and RB5, respectively. Experiments were carried out under solar light radiation with an average light intensity of 80,000 Lux. The disappearance of dye color using N-doped nanocatalysts without calcination is plotted as a function of irradiation time. As illustrated in FIG. 33, for 0.03 mmol/L of dye mixed with 10 mg/L Degussa P25 TiO₂, 53% of the dye color was removed within 1 hour and 95% decolorization was achieved in 3 hours. The decolorization rate was lower than that obtained with UV illumination under similar experimental conditions, as may be expected due to the absorption profile of the catalysts. It should also be noted that direct sunlight is a low intensity energy source, with an average intensity of about 80,000 Lux, which is several orders of magnitude lower than that used in the UV/TiO₂ experiments. The kinetics of dye decolorization is also presented in FIG. 33. Linear regressions show that all reactions followed apparent first-order kinetics. The overall reaction constants are 0.77 hr⁻¹ for AO7, 0.76 hr⁻¹ for MX-5B and 0.77 hr⁻¹ for RB5.

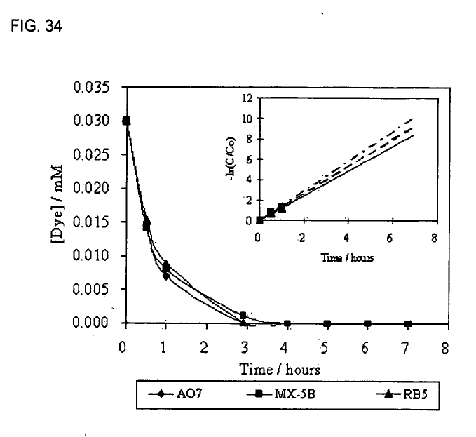

The decolorization of dye solutions using N-doped nanocatalysts with calcination is shown in FIG. 34. The decolorization in the dye solutions reached 70% within 1 hour, and 100% within 3 hours, with a catalyst loading of 10 mg L⁻¹. Three straight lines indicate that all reactions followed first-order kinetics. The slopes giving the apparent rate constants show that k′_{AO7} (1.44 hr⁻¹)>k′_{MX-5B} (1.36 hr⁻¹)>k′_{RB5} (1.24 hr⁻¹). The three dyes chosen for these experiments have similar molecular structures with the presence of an electron withdrawing SO₃⁻ group. However, they differ in terms of molecular weight, numbers of aromatic rings, numbers of azo bonds, and the presence or absence of halogen groups.

It is interesting to note that molecular structure had no significant effect on the photodegradation kinetics of selected dyes when nanocatalysts without calcinations were used, suggesting that the controlling reaction mechanism was related to the properties of the catalyst. By contrast, the differences in reaction constants with calcinated nanocatalyst-assisted photocatalysis indicate a direct relationship between molecular structure and photoreactivity. AO7 was the most reactive substance, which is probably due to it having the lowest molecular weight and its lack of halogen atoms. While neither desiring to be bound by any particular theory, nor intending to limit in any measure the scope of the appended claims or their equivalents, it is presently believed that the lower decolorization rate of MX-5B and RB5 could be due to reduced adsorption of these dyes on the catalyst surface caused by their larger sizes. MX-5B has two Cl groups bound to the aromatic ring system, which deactivates the molecule with respect to electrophilic degradation. The presence of two azo bonds in RB5 is likely another reason why RB5 exhibited the lowest decolorization efficiency.

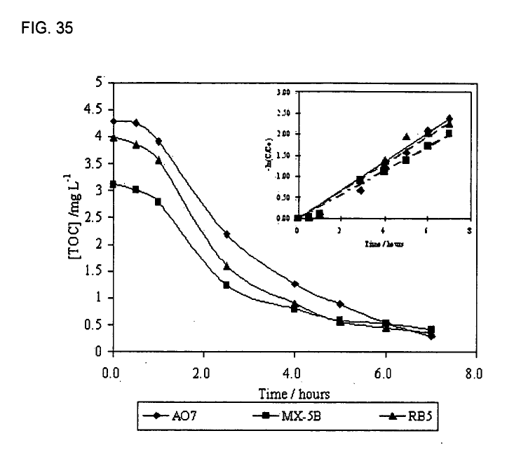

The kinetics of TOC disappearance were examined for the nanocatalysts after calcination. As shown in FIG. 35, for samples containing 0.03 mmol/L azo dyes mixed with 10 mg/L N-doped TiO₂ nanoparticles and illuminated by solar light with an average intensity of 80,000 Lux, all three dyes followed the same pattern showing about 8% of the TOC degraded during the first hour and more marked degradation during the second hour of the reaction with over 60% TOC reduction. About 80% of the carbon disappeared after 4 hours of sunlight irradiation. All reactions followed apparent first-order kinetics verified by the linear regression of f(t)=−ln(C/Co). The reaction constant for the three dyes are quite similar with k_{RB5} (0.34 hr⁻¹)>k_{AO7} (0.32 hr⁻¹)>k_{MX-5}B (0.28 hr⁻). These reaction rates are much lower than those for decolorization, suggesting that the breakage of the azo bond is the first step in dye degradation. The relatively slow TOC reduction was most likely caused by the transformation of parent compounds into smaller organic intermediates, such as acetic acids, phenols, aldehydes, and the like, which still contribute to the TOC of the solution. The primary breakdown products then undergo further oxidation leading to the production of CO₂. The presence of a low but constant level of TOC in solution after extended irradiation suggests the accumulation of photocatalytic reaction end products that are not completely mineralized to water and CO₂ during that time.

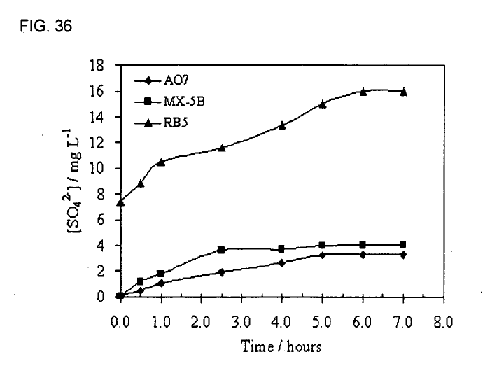

During the course of dye degradation, the inorganic anion SO₄²⁻ was formed progressively. As shown in FIG. 36, for samples containing 0.03 mmol/L azo dyes mixed with 10 mg/L N-doped TiO₂ nanoparticles and illuminated by solar light with an average intensity of 80,000 Lux, the amount of SO₄²⁻ increased more than 30% per hour, which is faster than the TOC disappearance rate and slower than the decolorization rate. RB5 shows a 7.43 mg/L initial sulfate ion concentration, which may be explained by an impurity of RB5.

The effect of N-doped TiO₂ nanoparticles loading on the AO7 decolorization rate has also been studied. Three different loadings (10 mg L⁻¹, 20 mg L⁻¹ and 50 mg L⁻¹) were used in this experiment, with initial dye concentrations of 0.06 mM. Sharp increases in dye degradation rate were observed when the nanocatalyst loading was changed from 10 mg L⁻¹ to 20 mg L⁻¹. On the other hand, only a slight enhancement was observed when the nanocatalyst concentration was further increased to 50 mg L⁻¹. This suggests that adding too much nanocatalyst into a dye solution may simply lead to saturation at low light levels without further increasing the catalytic performance. While neither desiring to be bound by any particular theory, nor intending to limit in any measure the scope of the appended claims or their equivalents, another explanation may be that the N-doped nanocatalyst loading caused a light screening effect and, therefore, reduced the photoactivity of the nanocatalyst at elevated concentrations.

A further application of doped metal oxide nanoparticles embodying features of the present invention is their use as photocatalysts for providing “self-sterilizing” surfaces, surface coatings, and the like. By way of example, a doped metal oxide nanoparticles embodying features of the present invention may be added to a composition for covering a surface (e.g., paints, lacquers, varnishes, sealants, and the like) to serve as a catalyst for degrading environmental contaminants including but not limited to dirt, bacteria, fungi, viruses, spores, toxins, chemical warfare agents, biological warfare agents, and the like that may come in contact with the surface.

A composition for covering a surface embodying features of the present invention includes a solvent, one or a plurality of pigments, and one or a plurality of titanium dioxide nanoparticles containing a non-metallic dopant selected from the group consisting of boron, carbon, silicon, germanium, nitrogen, phosphorous, arsenic, sulfur, selenium, tellurium, fluorine, chlorine, bromine, iodine, and combinations thereof. In some embodiments, at least a portion of the nanoparticles absorb visible light, which is particularly useful in applications for preventing or minimizing the growth and/or accumulation of environmental contaminants on the exterior surface of a building and/or on interior surfaces for which sterility is desirable (e.g., the walls of a surgical operating room or the like). In some embodiments, the nanoparticles absorb light in the yellow to orange range, which is a particularly acceptable color range for use in paints, coatings, and the like. In some embodiments, the non-metallic dopant is selected from the group consisting of carbon, silicon, nitrogen, phosphorous, sulfur, fluorine, chlorine, and combinations thereof.

A further application of doped metal oxide nanoparticles embodying features of the present invention is their use as photocatalysts for chemical reactions.

A method for catalyzing a chemical reaction with sunlight embodying features of the present invention includes (a) providing one or a plurality of titanium dioxide nanoparticles containing a non-metallic dopant selected from the group consisting of boron, carbon, silicon, germanium, nitrogen, phosphorous, arsenic, sulfur, selenium, tellurium, fluorine, chlorine, bromine, iodine, and combinations thereof; (b) activating at least a portion of the nanoparticles with sunlight to form activated nanoparticles; and (c) catalyzing a chemical reaction with the activated nanoparticles. In some embodiments, the chemical reaction is a transformation selected from the group consisting of conversion of hydrogen gas into protons, conversion of protons and oxygen into water, and a combination thereof. In some embodiments, the chemical reaction occurs in a fuel cell.

A further application of doped metal oxide nanoparticles embodying features of the present invention is their use as reagents in medical applications including but not limited to photodynamic therapies.

A method of treating a patient having cancer embodying features of the present invention includes (a) providing one or a plurality of titanium dioxide nanoparticles containing a non-metallic dopant selected from the group consisting of boron, carbon, silicon, germanium, nitrogen, phosphorous, arsenic, sulfur, selenium, tellurium, fluorine, chlorine, bromine, iodine, and combinations thereof; (b) activating at least a portion of the nanoparticles with light to form activated nanoparticles; (c) transferring energy from at least a portion of the activated nanoparticles to an oxygen molecule to form a reactive singlet oxygen; and (d) reacting the reactive singlet oxygen with a cancer cell thereby destroying the cancer cell.

The following representative procedures and examples are provided solely by way of illustration, and are not intended to limit the scope of the appended claims or their equivalents.

Synthesis of Nitrogen-Doped TiO₂ Nanocrystals by Hydrolysis Method

A 5-mL aliquot of Ti[OCH(CH₃)₂]₄ (Aldrich, 97%) dissolved in isopropyl alcohol (5:95) was added dropwise (1 mL/min) to 900 mL of doubly distilled water (2° C.) adjusted to pH 2 by HNO₃ addition. After continuous stirring of the reaction mixture for 12 h, a colloidal solution of TiO₂ nanocrystals was formed. By controlling the amount of added nitric acid, TiO₂ nanoparticles having sizes ranging from 3 to 10 nm may be synthesized and are stable for extended periods of time under refrigeration. Treatment of the initial nanoparticle solution and gel with an excess of triethylamine resulted in the formation of a yellowish solution. The solution was treated directly with an alkyl ammonium compound to facilitate nitrogen incorporation. Upon vacuum drying (5×10⁻² Torr) for several hours, the treated nanoparticle solution formed deep-yellow crystallites.

Synthesis of Nitrogen-Doped TiO, Nanocrystals by Oxidation Method

About 0.5 g of TiX powder is loaded in a ceramic sample boat (e.g., 4.0/l×0.5/h×0.5/w in inch), which is then placed in the middle of a quartz tube in a Lindberg tube furnace. The two ends of this tube are left open to the atmosphere. The temperature is slowly ramped up at a rate of 2° C./min, maintained at 300° C. to 650° C. for 96 hrs, and cooled to room temperature naturally. The samples are then taken out for further investigations.

Procedures for Investigating the Electronic Structure of Visible Light Responsive Doped TiO₂ Nanocrystals

Titanium nitride (TiN), titanium carbide (TiC) and titanium sulfide (TiS₂) were purchased from Strem Chemicals. The resultant titanium dioxide samples were prepared by heating the above-identified reagents at 1000° C. for 6 hours in a quartz tube under the atmosphere inside a Lindberg tube furnace with a digital control unit. The samples were then cooled, and all measurements were made at room temperature.

XRD patterns were obtained for the different nanocrystal samples using a Philips PW 3710 X-ray powder diffractometer. The UV-visible reflectance spectra of the nanocrystal samples were measured on a Cary 50 UV-visible spectrometer with a fiber optical reflectance unit.

The X-ray spectroscopic experiments were measured at the undulator beamline 7.0 of the ALS, Lawrence Berkeley National Laboratory with a spherical grating monochromator. Ti L_{2,3} XES and O K_{α} XES spectra of these samples were recorded using the Nordgren-type grating spectrometer. The spectrometer was mounted perpendicular to the incoming photon beam in the polarization plane, and the resolution was 0.3 eV and 0.4 eV, respectively, for the Ti L_{2,3} XES and O K_{α} XES spectra. The monochromator was set to the excitation energies of 475 eV and 565 eV, respectively, for Ti L_{2,3} and O K_{α} spectra with a resolution of 0.5 eV. The samples were mounted to have beam in a 300 incidence angle to sample surface. For energy calibration of Ti L_{2,3} XES and O K_{α} XES, the spectra of reference samples Ti, TiO₂, and Zn were measured. The base pressure of the chamber was 2×10⁻⁹ mbar. The absorption spectra at the Ti 2p and O 1s edges were measured by means of total electron yield and with a monochromator resolution set to 0.2 eV. The absorption intensity was normalized by the current from a clean gold mesh in the incoming beam to eliminate the fluctuations of incoming photon intensity. The x-ray fluorescence and absorption spectra were brought to a common energy scale using an elastic peak in the fluorescence spectra recorded at the excitation energy set at the absorption edge.

For the XPS measurements, a Perkin-Elmer PHI 5600×PS System was used. The energy resolution of the spectrometer is 0.3-0.5 eV for the XPS measurements. Samples for XPS measurement were coated on carbon tape attached to the sample holder. The pressure in the vacuum chamber during the measurements was below 3×10⁻⁸ mbar.

Procedures for the Photocatalytic Degradation of Azo Dyes

The nitrogen-doped titanium dioxide nanocatalysts used for the photocatalytic degradation of azo dyes were prepared by adding very slowly 9 ml of Ti[OCH(CH₃)₂]₄ (Aldrich, 97%) to 150 ml ethanol and 5 ml aqueous ammonia solution. After continuous stirring of the reaction mixture at 85° C. for 4 hours, a colloidal solution of TiO₂ nanocrystals was formed. After centrifugation and removal of the solvent under vacuum for several hours, gray powders were obtained. After calcination at 400° C. for 3 hours, a light-yellow powder was obtained. The nanocatalysts were characterized by XRD using a Philips PW 3710 X-ray powder diffractometer, and by TEM using a JEOL 1200EX transmission electron microscope operated at 80 kV. Samples for TEM analysis were prepared by depositing a drop of a nanocrystal solution in water onto a copper grid supporting a thin film of amorphous carbon. The grid was dried in the air.

Acid orange 7, procion red MX-5B, and reactive black were obtained from Sigma-Aldrich and were used as received without further purification. All aqueous dye solutions were prepared with water from a Millipore Waters Milli-Q purification unit.

Both solar light and UV illumination were used as energy sources in this study. The UV light source was a 150 W high-pressure mercury lamp (UXL-151H) with a wavelength range greater than 250 nm and a maximum output between 250 and 450 nm. Experiments were carried out in Petri dishes with a 10-cm diameter. Each Petri dish contained 15 ml of a dye and TiO₂ suspension. The Petri dishes were covered with plastic films to prevent evaporation of the dye solution. Neither forced aeration nor stirring of the dye solution was conducted in these experiments. Experiments using solar light were carried out from 9 am to 5 pm during the summer season in Milwaukee, Wis. For each set of experiments, eight dishes were prepared and placed under direct sunlight. One of the Petri dishes was wrapped with aluminum foil at the end of each experimental period, and the solution was transferred into a 15-ml graduated tube covered with aluminum foil for further analysis.

A broad range LUX/FC luxmeter (Sper Scientific) was used to measure the light intensity for solar experiments. Decolorization of azo dyes was determined by examining the concentration of dyes using their maximum absorbance in a UV-vis spectrophotometer (Milton Roy, SPECTRONIC GENESYS). The concentration of sulfate (SO₄²⁻) ion was analyzed by ion chromatography (Dionex IC25). Total organic carbon of the dye solutions was measured using a TOC monitor (Shimadzu, TOC-5000).

The foregoing detailed description and accompanying drawings have been provided solely by way of explanation and illustration, and are not intended to limit the scope of the appended claims. Many variations in the presently preferred embodiments illustrated herein will be apparent to one of ordinary skill in the art, and remain within the scope of the appended claims and their equivalents.

1. A material comprising:
one or a plurality of titanium dioxide nanoparticles comprising a non-metallic dopant selected from the group consisting of boron, carbon, silicon, germanium, phosphorous, arsenic, sulfur, selenium, tellurium, fluorine, chlorine, bromine, iodine, and combinations thereof.

2. The invention of claim 1 wherein the non-metallic dopant is selected from the group consisting of boron, carbon, silicon, phosphorous, sulfur, selenium, fluorine, chlorine, bromine, and combinations thereof.

3. The invention of claim 1 wherein the non-metallic dopant is selected from the group consisting of carbon, silicon, phosphorous, sulfur, fluorine, chlorine, and combinations thereof.

4. The invention of claim 1 wherein an average diameter of the nanoparticles ranges from about 0.1 nm to about 1000 nm.

5. The invention of claim 1 wherein an average diameter of the nanoparticles ranges from about 0.3 nm to about 500 nm.

6. The invention of claim 1 wherein an average diameter of the nanoparticles ranges from about 0.5 nm to about 350 nm.

7. The invention of claim 1 wherein an average diameter of the nanoparticles ranges from about 1 nm to about 200 nm.

8. The invention of claim 1 wherein the nanoparticles comprise from about 0.1 percent to about 15 percent of the non-metallic dopant.

9. The invention of claim 1 wherein the nanoparticles comprise from about 0.5 percent to about 12 percent of the non-metallic dopant.

10. The invention of claim 1 wherein the nanoparticles comprise from about 1 percent to about 10 percent of the non-metallic dopant.

11. The invention of claim 1 wherein the nanoparticles absorb visible light.

12. The invention of claim 1 wherein the nanoparticles absorb light having a wavelength of at least about 390 nm.

13. The invention of claim 1 wherein the nanoparticles absorb light having a wavelength of at least about 450 nm.

14. The invention of claim 1 wherein the nanoparticles absorb light having a wavelength of at least about 500 nm.

15. The invention of claim 1 wherein the nanoparticles absorb light having a wavelength of at least about 550 nm.

16. The invention of claim 1 wherein the nanoparticles further comprise a metal cap on at least a portion of an outer surface thereof.

17. The invention of claim 16 wherein the metal cap comprises a transition group metal.

18. The invention of claim 17 wherein the transition group metal is selected from the group consisting of ruthenium, rhodium, nickel, palladium, platinum, copper, gold, silver, and combinations thereof.

19. The invention of claim 1 wherein the nanoparticles further comprise a metallic dopant.

20. The invention of claim 19 wherein the metallic dopant is a transition metal.

21. A material comprising:
one or a plurality of titanium dioxide nanoparticles comprising a non-metallic dopant selected from the group consisting of boron, carbon, silicon, germanium, phosphorous, arsenic, sulfur, selenium, tellurium, fluorine, chlorine, bromine, iodine, and combinations thereof;
wherein the nanoparticles comprise an average diameter ranging from about 0.5 nm to about 350 nm;
wherein the nanoparticles comprise from about 0.1 percent to about 15 percent of the non-metallic dopant; and
wherein at least a portion of the nanoparticles absorb visible light.

22. The invention of claim 21 wherein the nanoparticles further comprise a metal cap on at least a portion of an outer surface thereof, and wherein the metal cap comprises a transition group metal.

23. The invention of claim 21 wherein the nanoparticles further comprise a metallic dopant.

24-43. (canceled)

44. A doped titanium dioxide nanoparticle comprising:
a core portion, a shell portion, and a non-metallic dopant;
wherein the core portion is adjacent to a center of the nanoparticle and the shell portion is adjacent to an exterior surface of the nanoparticle;
wherein the non-metallic dopant is selected from the group consisting of boron, carbon, silicon, germanium, phosphorous, arsenic, sulfur, selenium, tellurium, fluorine, chlorine, bromine, iodine, and combinations thereof; and
wherein a concentration of the non-metallic dopant is higher in the shell portion than in the core portion.

45. (canceled)

46. A doped titanium dioxide nanoparticle comprising:
a core portion, a shell portion, and a non-metallic dopant;
wherein the core portion is adjacent to a center of the nanoparticle and the shell portion is adjacent to an exterior surface of the nanoparticle;
wherein the non-metallic dopant is selected from the group consisting of boron, carbon, silicon, germanium, nitrogen, phosphorous, arsenic, sulfur, selenium, tellurium, fluorine, chlorine, bromine, iodine, and combinations thereof; and
wherein a concentration of the non-metallic dopant is higher in the core portion than in the shell portion.

47. The invention of claim 46 wherein the non-metallic dopant is selected from the group consisting of carbon, nitrogen, sulfur, and phosphorous.

48. The invention of claim 46 wherein the non-metallic dopant comprises nitrogen.

49-60. (canceled)
